SEQUENCE LISTING (39 sequences)

SEQ ID NO: 1 - protein, 359 aa, Aggregatibacter actinomycetemcomitans
CVKGNSIHPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGIKYLQGLKSRQVDDEIDITNADSIAFMQSLMSEVIDIFGDTSQHFHIGGDEFGYSVESNHEFITYANKLSYFLEKKGLKTRMWNDGLIKSTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKANGDE

SEQ ID NO: 2 - protein, 346 aa, Haemophilus sputorum
QNSTKQSGLMLDISRRFYSVETIKQFIDDIAQANGTFLHLHFADHENYALESTFLNQRAENAIVQNGIYINPKTNKPFLTYEQIDQIIRYAQEKQIELIPEVDSPAHIKGILTLLRLEKGEDYVNQIALNQDELNLDSPESLTMMKTLVDEVCYIFGYSAQHFHIGGDEFNYASNFIRYVNALNQHINQKGLITRMWNDGLLQQNIDELDKNIEITYWSFDGDAQEKNDIVERRATRISLPTLLDKGFKALNYNSYYLYFIPKDNGNIATDAKFALNDLKQNWQLLRWDGNYETQPIQQAENLIGAAFSIWGEHAGKLSDDVIHQATSPLIQATIIQTNAKTTGPN

SEQ ID NO: 3 - protein, 352 aa, Actinobacillus suis
MNHSQIKEAGLTLDIARRFYPVETIKQFIDTIHHAGGTFLHLHFSDHENYALESTYLDQSEANAIVKDGTYYNPKTNKPFLTYKQIHDIIYYAKSKNIELVPEVDTPNHMTAIFRLLEAKHGKDYVKKLKSKMNDEEIDITNPESIEVIKTLIAEVIYIFGHASEHFHIGGDEFGYSVETNHEFISYVNTLNQFINEKGKITRIWNDGLIKNNLNQLNKNVEITYWSYDGDAQESQDIAERRKIRANLPELLENGFKVLNYNSYYLYFVPKGNANITHDSKYATEDVLNNWKLGLWDGQNKENMVENTKNIIGSSLSIWGERSGSLSSEVIEESTQDLLKAVIQKTNDPKSH

SEQ ID NO: 4 - protein, 352 aa, Actinobacillus capsulatus
MNHSQIKEAGLTLDIARRFYPVETIKQFIDTIHHAGGTFLHLHFSDHENYALESTYLDQLEANAIVKDGTYYNPTTNKPFLTYKQINDIIYYAKSKNIELVPEVDTPNHMTAIFRLLEAKHSKDYVKRLKSKMNDEEIDITNLESIEVIKTLIAEVIYIFGHASEHFHIGGDEFGYSVETNHEFITYVNTLNQFINNKGKITRIWNDGLIKNNLNQLNKNVEITYWSYDGDAQESQDIAERRKIRVNLPELLENGFKVLNYNSYYLYFVPKGNANITHDSKHATEDVLKNWKLGLWDGQNKENIVENTKNIIGSSLSIWGEHSGSLSSAVIEESTQELLKAVIQKTNDPKSH

SEQ ID NO: 5 - protein, 352 aa, Actinobacillus equuli
MNHSQIKEAGLTLDIARRFYPVETIKQFIDTIHHAGGTFLHLHFSDHENYALESSYLDQSEENAIVKDGTYYNPKTNKPFLTYKQIDDIIYYAKSKNIELVPEVDTPNHMTAIFNLLEIKHGEAYVKNLKSKMNDEEIDITNPESIEVIKTLIAEVIYIFGHASEHFHIGGDEFGYSVETNHEFISYVNTLNQFINEKGKITRIWNDGLIKNNLNQLNKNVEITYWSYDGDAQKSQDIAERRKIRADLPELLENGFKVLNYNSYYLYFVPKGNANITHDSKYATEDVLNNWKLGLWDGKNKENEVKNTKNIIGSSLSIWGERSGSLSSEVIEESTQDLLKAVIQKTNDPKSH

SEQ ID NO: 6 - protein, 359 aa, Aggregatibacter actinomycetemcomitans
CVKGNSIYPQKISTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSIAFMQSLMNEVIDIFGDTSQHFHIGGDEFGYSVESNHEFITYANKLSYFLEKKGLKTRMWNDGLIKSTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 7 - protein, 359 aa, Aggregatibacter actinomycetemcomitans
CVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 8 - protein, 351 aa, Actinobacillus pleuropneumoniae
MDLPKKESGLTLDIARRFYTVDTIKQFIDTIHQAGGTFLHLHFSDHENYALESSYLEQREENATEKNGTYFNPKTNKPFLTYKQLNEIIYYAKERNIEIVPEVDSPNHMTAIFDLLTLKHGKEYVKGLKSPYIAEEIDINNPEAVEVIKTLIGEVIYIFGHSSRHFHIGGDEFSYAVENNHEFIRYVNTLNDFINSKGLITRVWNDGLIKNNLSELNKNIEITYWSYDGDAQAKEDIQYRREIRADLPELLANGFKVLNYNSYYLYFVPKSGSNIHNDGKYAAEDVLNNWTLGKWDGKNSSNHVQNTQNIIGSSLSIWGERSSALNEQTIQQASKNLLKAVIQKTNDPKSH

SEQ ID NO: 9 - protein, 461 aa, Curtobacterium oceanosedimentum
ADRNTSAAEAAVTSIAPRATITGVAAISAATSSRTTVRTTLTLENRSGERESAADAWLYLAGGGARYALGHAPVRALAAGARATVRTTLRVPSRAPAGKYAVLACAGPYSKQACRTSGTTVTVGTAARARPETGVMLDVARAYYPVSLIEQYVDLLAEHGGGFLHLHLTDDQNVGIESAVLGQTPANAVLRNGVYTSRVTGRPFLSAAQARAISAYAAKRGIAIVPEVDSPGHMAAAFALLEARHGATWVDRIRSGESELDTSVPESATLAAELLREVTQTFPSSRTVHIGGDEWGADVSADERVGWMNAMAAAIGDREVWAWNDGIDRASVGRLDPRIHVTYWSFDGDTEDAAERRERRARRASATDLQRAGIDLLNYNSYYLYEVPTDLDPADSEYTVADLREHWSLRAWDGDSGARLAAPMSGAAVAIWGEDLDGAPSEALLRWSAPHVTAMIETAAS

SEQ ID NO: 10 - protein, 445 aa, Curtobacterium flaccumfaciens
DTAVSAVTVTKVTASTTGTVVRTTLKVENTAPVRKPASSVWLYLSAGTEKYTLGRVAVKALAAGSSTSVTAVRGTPSRAAAGKYSVLACAGAYSAKQCRTSTATVTTKPTKRARPETGVMLDVARAYYPVALIKRYIDLLADDGGRFLHLHLTDDQNVGIESTVLGQTPANADLDHGVYTSRVTHRPFLSAAQARTISAYGAERGVAIVPEIDTPGHMAAAFALLEAQHGTKWVDRIRSGENELDTSAPESLALAKKLYAEVQRTFPSSRTVHIGGDEWGDDVTAAQRVTWMNAMAAALDDREVWAWNDGIDRVAVGRLDPRIHVTYWSFDGDTEDAAERRERRARRASAVDLQQAGIDQLNYNSYYLYEVPTDLDPADSDYTVADLRENWSLRAWDGDSGSLLAAPMSGAAVAIWGEDLEDPPSDALLRWSAPHVTAMIETAAS

SEQ ID NO: 11 - protein, 445 aa, Curtobacterium luteum
DTAVSAVTVTKVTASTTGTAVRTTLKVENTAPVRKPASSVWLYLSAGTEKYTLGRVAVKALSAGSSTSVTAVRGTPSRAAAGKYWVLACAGAYSAKQCRTSTATVTTKPTKRARPETGVMLDVARAYYPVALIKRYIDLLADDGGRFLHLHLTDDQNVGIESTVLGQTPANADLDHGVYTSRVTHRPFLSAAQARTISEYGAERGVTIVPEIDTPGHMAAAFALLEAQHGTKWVDRIRSGENELDTSAPESLVLAKKLYAEVQRTFPSSRTVHIGGDEWGDDVTAAHRVAWMNEMAATLGNREVWAWNDGIDRVAVGRLDPRIHVTYWSFDGDTEDAAERRERRARRASAVDLQQAGIDQLNYNSYYLYEVPTDLDPADSDYTVADLRENWSLRAWDGDSGSLLAAPMSGAAVAIWGEDLEDPPSDALLRWSAPHVTAMIETAAS

SEQ ID NO: 12 - protein, 458 aa, Curtobacterium oceanosedimentum
IGGSAGTADASGAPRLVVTKVTASSTTTSTRTTVRTTLTVKNTSVARKPAADAWLSLTAGSKRYTLGHVSVQSLAAGASATIHATHTAPPRAPAGKYAVLACTGAFSLSKCGTSATTVTTARATRARPDTGVMLDVARAYYPVALIEQYIALLADHGGRFLHLHLTDDQNVGIESEVLGQTLANADLRDGVYTSRITGRPFLSAAQAREISRYAAQRGIAIIPEIDTPGHMAAAFALLEAGHGKQWVDRIRSGESELDTSAPGSSALAARLLQEVTRTFPSSRTVHIGGDEWGDDVTADERVQWLNTMAAAVGNRAVWAWNDGIDRAAIGRLDPRIHVTYWSFDGDTEDATERRERRERRAGANDLYAAGIDLLNYNSYYLYEVPTDLDAADSEYTVADLRENWSLRTWDGDSGARLAGPTSGAAVAIWGEDLEAPPSDALLRWSAPHVLAMIETAGS

SEQ ID NO: 13 - protein, 450 aa, Curtobacterium leaf154
AGSTTSTVTVTQVTATTTASSTGTAVRTTLKIKNTAAVRKPASSAWLYLSAGTKKYTLGRVAVKALAAGSSTSVTAVRGTPSRATAGEYSVLACAGAYSAKQCRTSTATVTTKPTKRARPETGVMLDVARAYYPVALIKRYIDLLADDGGRFLHLHLTDDQNVGIESTVLGQTLANADLDEGVYTSRVTRRPFLSAAQARTISDYAARRGVAIVPEIDTPGHMTAAFDLLEAQHGTKWVDRIRSGENELDTSTPGSLALAKKLYAEVQRTFPASRTVHIGGDEWGDDVSAAERVAWMNAMAAALGNREVWAWNDGIDRVAVGRLDPRIHVTYWSFDGDTEDAAERRERRARRASAVDLQQAGIDMLNYNSYYLYEVPTDLDPADSEYTVADLRENWSLRTWDGDSGSLLAAPMSGAAVAIWGEDLEDPPSDALLRWSAPHVTAMIETAAS

SEQ ID NO: 14 - protein, 324 aa, Terribacillus saccharophilus
QDQEKGITIDISRKYYSIKTLKAIVDEISANGGDYLQLHFSDNESYAIASEFLGQNSENPNSAYLTKKELLSLIAYSNDRNIMVIPDIDLPAHSKGWLNIMKEKDSGLYTDIVTDYSEDTLDYHNNAVALYTANQLLDEVLDLFYQPKFAGKQRIVLGGDEVPGSGAHQTDFIRFMNQIAKTAKASNYEPQMWNDSITPEGIQNLDRSFSILYWKQSTLSNGAQSLDVQDFEENGLSVYNYNAYSLYFLPSTRFTQEDITEQIDYMKWAYAYNKFFYISDYYKQVDTPNVKGSSLVFWGEHANDLSQEGLLKQEKPLIQNFLGL

SEQ ID NO: 15 - protein, 324 aa, Terribacillus goriensis
QDQEKGITIDISRKYYSIETLKSIIDEISANGGDYLQLHFSDNERYAIASEFLGQNGENPNSTYLTKKELLSLIAYSNDRDIMVIPDIDLPAHSRGWLNIMKEKDSGLYTDIVTDYSEDTLDYHNNAVALYTANQLLDEVLDLFYQPKFAGKQRIVLGGDEVPGSGVHQTDFIRFMNQIAETAKASNYKPQMWNDSITPEGIQNLDRSFSILYWKQSTLSNGAQGLDVQDFEENGLSVYNYNAYSLYFLPATRFTQEDITEQIDYMKWAYAYNKFFYISDYYKQVDTSNVKGSSLVFWGEHANDLSQEGLLKQEKPLIQNFLGL

SEQ ID NO: 16 - protein, 324 aa, Terribacillus saccharophilus
KDQEKGITIDISRKYYSIGTLKAIVDEINANGGDYLQLHFSDNESYAIASEFLGQNSENPNSTYLTKKELLSLIAYSNDRNIMVIPDIDLPAHSKGWLNVMKEKDSGLYTDIVTDYSEDTLDYHNNAAALYTANQLLDEVLDLFYQPKFAGKQRIVLGGDEVPGSGAHQTDFIRFMNQIDETAKASNYEPQMWNDSITPEGIQNLDRSFSILYWKQSTLSSGAQGLDVQNFEEKGFSVYNYNAYSLYFLPSTRFTQEDITEQIDYMKWAYAYNKFFYISDYYKQVDTSNVKGSSLVFWGEHANDLSQEGLLEQEKPLIQNFLSL

SEQ ID NO: 17 - protein, 324 aa, Terribacillus saccharophilus [sequence omitted]

SEQ ID NO: 18 - protein, 324 aa, Terribacillus saccharophilus
KDQEKGISIDISRKYYSIGTLKAIIDEISANGGDYLQLHFSDNESYAIASDYLGQISDTPNNTYLTKNDLLSLIAYSNDRNILIIPDMDLPAHSRGWLELMKVKDRELYTDIVTDYSNETLDYHNNTDALNTANQLLNEILELFYQPKFAGKQRIVLGGDEVPGSEIHQLDFIRFINQIASTAKASNYAPQMWNDSITAEGIQNLDKSFSILYWKQSTLSNGAQSLEVQDFEDWDFPVYNYNAYSLYFLPSIRFTDEDITEQMNYMKWAYAYNKFFYISDYYKSVDASNVKGSSLTFWGEHATDLSQEELLEQELPLIKKFLSL

SEQ ID NO: 19 - protein, 331 aa, Lactobacillus paraplantarum
NSSTLNTSQGVMLDLGRHPLDETAIKAVISAAAEQHMQYVELHLSDNEHLCFQSAYLGNAASATVLSATTLEQLVAYANQLNIELVPDVDLPSHAGAILRQLQQTHPDIYNTVKLDDETIDYTKPAAISLATTLYGELDASFNNQSQHDLMLGADEVPGSASAYIELTTFINQVSRFQNQHGFNTSIWNDSLLKNELTRLDSNITINYWSQSGNNTDVAIIADRYANRVSVPDILASGHPIVNCNSYATYYQIKNIGNVNDDDYFINYLNHTFRPNIFNEIDTNGHNQDWTIEDGVTTNGILVSLWGADSEHVTPTAIVNFIKRMTIPRSF

SEQ ID NO: 20 - protein, 353 aa, Lactobacillus apinorum
TLADTSNDTKRIGLSLDCSRTYYSPSTIKKYIDLLKKDHGTYLQLHLNDNERYGVESSTLGQTTQNATLKDGVYYNNKTHLAFLSKNQLLDVIQYGYTHGIEVIPEIDLPGHAQSIFKLLSYTSEGKKLVKELENKDGYNEMYYNKQATIDFSKKLLSEYVGMLPSGYHIIVGADEITISDKSDQEAVVKYINAIDDYVNANHLKLEMWNDSFHKAVLSKYHKDILINYWSLTGEVSSSKDRKDNIRMRATLPELNKAGFKTINYNSYYLYMITDPTSFTNESKKIWTSEFKKWKMNMWNDESTKDITKSANNIGAAISIWGEYPNQYTGDQTYNKTYYYVDTFLKAQDKFTK

SEQ ID NO: 21 - protein, 331 aa, Lactobacillus paraplantarum
NSSTLNTSQGVMLDLGRHPLDETAIKAVISAAAEQHMQYVELHLSDNEHLCFQSAYLGNAASATVLSATTLEQLVAYANQLNIELVPDVDLPSHAGAILRQLQQTHPDIYNTVKLDDETIDYTKPAAVSLATTLYGELDASFNNQSQHDLMLGADEVSGSASAYIELTTFINQVSRFQNQNGFNTSIWNDSLLKNELNRLDSNITINYWSQSGNNTDAAIIADRYANRASVPDILASGHPIVNCNSYATYYQFKNIGNVNDDNYFINYLNHTFRPNIFNEIDTNGHNQDWTIEDGVTTNGILVSLWGADSEHVTPTAIVNFIKRMAIPRSF

SEQ ID NO: 22 - protein, 323 aa, Staphylococcus cohnii
QDFQKGINVDIARKDYSLKSLKKIVDTIHENNGDYLQLHFSDNENYAIESQFFKHENIASQNYLSQQELKNLIHYSNKLNIMVVPEFDLPSHSKAWLLLLKNENSNLHENIVSDYSDETIDFFSNQKALEISKRQIKEILNLFHQPNFQKEQRIVLGGDEVPGGKSYQNDFINFMNEIGEYAYQNGYEPQIWNDSITKNGLKLLKNYFSVIFWKQSNNENNEPGITVEDFLDYNFKVYNYNFYSLYFLPSKNYSPTDIEEQTSYISWAYNHNSFYYLKNPYYEVDSLNIQGSALSFWGEHATGMREEEVLNQELPLIRTYLNK

SEQ ID NO: 23 - protein, 321 aa, Staphylococcus fleurettii
ESIQEGVSVDIARKEYSLESLKQIVDTIHENNGQYLQLHFSDDENYAIESDYFSHQGIPNENYLTKAEIKSLIAYSNELNVMVVPDIDFPSHSKALLSLIKNEDKDLYNQIISDYSDNTFDFFSNDKALAISKRHIGEITTLFNQPKYNGQQRIVLGGDEVPGGGAYQSDFISYMNNIGSYAAGQGYEPQMWNDMISHEGIKSLNDTFSILYWKQNENSKSDLTVEDFAEYDFKIYNYNFYSLYFLPSNQFTNADIEEQADYISWAYAYNKFFYTNEPYQEVDSDNVKGSALSFWGEDALNMSQTELINQEIPLIKAYFSS

SEQ ID NO: 24 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 25 - protein, 269 aa, Artificial
  Bacillus lentus protease variant
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASLNGTSMATPHVAGAAALVKQKNPSWSNVQIRNHLKNTATSLGSTNLYGSGLVNAEAATR

SEQ ID NO: 26 - protein, 269 aa, Artificial
  Bacillus lentus protease variant
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGDGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASLNGTSMATPHVAGAAALVKQKNPSWSNVQIRNHLKNTATSLGSTNLYGSGLVNAEAATR

SEQ ID NO: 27 - protein, 269 aa, Artificial
  Protease variant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 28 - protein, 275 aa, Bacillus amyloliquefaciens [sequence omitted]

SEQ ID NO: 29 - protein, 269 aa, Bacillus lentus; identical to SEQ ID NO: 24

SEQ ID NO: 30 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 31 - protein, 269 aa, Artificial
  Protease variant
QQTVPWGITRVQAPTVHNRGITGSGVKVAILDTGIAQHSDLTVRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 32 - protein, 269 aa, Artificial
  Protease variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANLSLGSDAPSTTLERAVNYATSRGVLVIAATGNSGAGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 33 - protein, 269 aa, Artificial
  Protease variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASNDGTSMATPHVAGVAALVKQKNPSWNATQIRNHLKNTATNLGNSSQFGSGLVNADAATR

SEQ ID NO: 34 - protein, 269 aa, Artificial
  Protease variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASLDGTSMATPHVAGVAALVKQKNPSWNATQIRNHLKNTATNLGNSSQFGSGLVNADAATR

SEQ ID NO: 35 - protein, 269 aa, Artificial
  Protease variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSISYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 36 - protein, 269 aa, Artificial
  Protease variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSPAPSTTLERAVNYATSRGVLVIAATGNNGTGSIGYPARYANAMAVGATDQNNRRASFSQYGTGIDIVAPGVGIQSTYLNNSYASLDGTSMATPHVAGVAALVKQKNPSWNATQIRNHLKNTATNLGNSSQFGSGLVNADAATR

SEQ ID NO: 37 - protein, 269 aa, Artificial
  Protease variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMHGTSMATPHVAGVAALVKQKNPSWNATQIRNHLKNTATNLGNSSQFGSGLVNADAATR

SEQ ID NO: 38 - protein, 269 aa, Artificial
  Protease variant
QQTVPWGITRVLAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANLSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVSGVAALVKQKNPSWNATQIRNHLKNTATNLGNSSQFGSGLVNADAATR

SEQ ID NO: 39 - protein, 269 aa, Bacillus lentus; identical to SEQ ID NO: 24

(19) United States
(12) Patent Application Publication
(10) Pub. No.: US 2023/0024242 A1
(43) Pub. Date: Jan. 26, 2023

(54) CLEANING COMPOSITIONS COMPRISING DISPERSINS VIII

(71) Applicant: Henkel AG & Co. KGaA, Duesseldorf (DE)

(72) Inventors: Mirko WEIDE, Duesseldorf (DE); Susanne WIELAND, Zons/Dormagen (DE); Brian LAUFS, Juechen (DE); Michael KANDZIA, Solingen (DE); Ulrike DENGUTH, Oer-Erkenschwick (DE); Christian DEGERING, Erkrath (DE); Rune Nygaard MONRAD, Bagsvaerd (DK); Rebecca VEJBORG, Bagsvaerd (DK); Jesper SALOMON, Bagsvaerd (DK); Dorotea Raventos SEGURA, Bagsvaerd (DK)

(21) Appl. No.: 17/786,561

(22) PCT Filed: Dec. 7, 2020
(86) PCT No.: PCT/EP2020/084950

§ 371 Date: Jun. 17, 2022

(30) Foreign Application Priority Data
Dec. 20, 2019 (DE) 10 2019 135 357.2

(51) Int. Cl. C11D 3/386; C11D 11/00; C11D 3/00; C11D 3/43; C11D 3/08; C11D 3/36; C11D 3/22; C11D 3/37

(52) U.S. Cl. CPC C11D 3/38636; C11D 11/0017; C11D 3/0036; C11D 3/43; C11D 3/001; C11D 3/08; C11D 3/361; C11D 3/225; C11D 3/373; C11D 3/3769; C12Y 302/01052

(57) ABSTRACT

Cleaning compositions may include a mix of enzymes that include dispersins and proteases. Said compositions may be used in cleaning processes and/or for deep cleaning of organic stains, and for removal or reduction of components of organic matter.

### REFERENCE TO A SEQUENCE LISTING SUBMITTED VIA EFS-WEB

The content of the ASCII text file of the sequence listing named “2019P00544WOUS_P83439_seqlist_ST25_final”, which is 109 kb in size was created on Dec. 20, 2019. The sequence listing was corrected on Jun. 15, 2022 to correctly define the source of the genetic material; the corrected sequence listing is electronically submitted via EFS-Web herewith; the original sequence listing and the corrected sequence listing are incorporated by reference in their entirety.

### CROSS-REFERENCE TO RELATED APPLICATIONS

The present application is a national stage entry according to 35 U.S.C. § 371 of PCT application No.: PCT/EP2020/084950 filed on Dec. 7, 2020; which claims priority to German patent application 10 2019 135 357.2, filed on Dec. 20, 2019; all of which are incorporated herein by reference in their entirety and for all purposes.

### TECHNICAL FIELD

The present invention relates to compositions such as cleaning compositions comprising a mix of enzymes, as defined herein. The invention further relates to the use of said compositions comprising such enzymes in cleaning processes and/or for deep cleaning of organic stains, and methods of using said compositions for removal or reduction of components of organic matter.

### BACKGROUND

Enzymes have been used in detergents for decades. Usually a cocktail of various enzymes is added to detergent compositions, wherein each enzyme targets a specific substrate, e.g. amylases are active towards starch stains, proteases on protein stains and so forth. The effectiveness of these commercial enzymes provides detergents which remove much of the soiling. However, components of organic matters such as biofilm and EPS (extracellular polymeric substance) constitute a challenging type of staining due to the complex nature of such organic matter, and commercially available cleaning compositions are not able to effectively remove or reduce EPS and/or biofilm related stains. Textile surfaces and hard surfaces, such as dishes or the inner space of a laundry machine enduring a number of wash cycles, become soiled with many different types of soiling which may compose of proteins, grease, starch etc. One type of stain may be associated with organic matter such as biofilm, EPS (extracellular polymeric substance), etc. Organic matter may be composed of different molecules such as polysaccharides, extracellular DNA (eDNA), and proteins. Some organic matter comprises an extracellular polymeric matrix, which may be sticky or gluing, which when present on textile attracts soils and may cause redeposition or backstaining of soil, resulting in a greying of the textile. Additionally, organic matters such as biofilms often cause malodor issues as various malodor molecules can be adhered by the polysaccharides, extracellular DNA (eDNA) and proteins in the complex extracellular matrix and be slowly released to cause a noticeable malodor.

There is thus still a need for cleaning compositions which effectively prevent, reduce or remove stains e.g. associated with biofilms, such as protein and polysaccharides, e.g. PNAG (poly-β(1-6)-N-acetylglucosamine). The present invention provides new compositions fulfilling such need.

### SUMMARY

A first aspect of the present invention relates to a cleaning composition comprising a dispersin and a protease and, optionally, at least one cleaning component, wherein the protease is selected from

(1) a protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprising the amino acid substitution R99E in combination with at least two, preferably all three further amino acid substitutions selected from the group consisting of S3T, V4I and V199I, wherein positional numbering is according to SEQ ID NO:24; or
(2) a protease comprising an amino acid sequence having at least 90% sequence identity to SEQ ID NO:27, preferably at least 95% sequence identity, preferably at least 96% sequence identity, preferably at least 97% sequence identity, preferably at least 98% sequence identity to SEQ ID NO:27, wherein the protease variant has a glutamic acid residue (E) in position 101, and wherein the protease variant further comprises one or more substitutions selected from S156D; L262E; Q137H; S3T; R45E,D; P55N; T58W,Y,L; Q59D,M,N,T; G61 D,R; S87E; G97S; A98D,E,R; S106A,W; N117E; H120V,D,K,N; S124M; P129D; E136Q; S143W; S161T; S163A,G; Y171L; A172S; N185Q; V199M; Y209W; M222Q; N238H; V244T; N261T; and L262N,Q,D; or
(3) a protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:30 over its entire length and comprising an amino acid substitution in at least one position corresponding to positions 12, 43, 122, 127, 154, 156, 160, 211, 212 und 222 of SEQ ID NO:30: or
(4) a protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4%, or 99.5% sequence identity to the amino acid sequence set forth in SEQ ID NO:39 over its entire length and comprising (i) at least two of the amino acid substitutions 3T, 4I, 99E and 199I at the positions corresponding to positions 3, 4, 99 and 199 of SEQ ID NO:39 and (ii) in at least one position corresponding to positions 74, 136, 143, 154, 161, 163, 171, 200, 203, 209, 212 or 256 of SEQ ID NO:39 at least one amino acid substitution, preferably at least one amino acid substitution selected from 74D, 74E, 74Q, 136Q, 143L, 143W, 143Y, 154D, 154Q, 161T, 163G, 171L, 200A, 200L, 200S, 200T, 203K, 203V, 203W, 209W, 212N, 212S, 212T, 256D, 256E or 256Q.

In various embodiments, the cleaning composition of the invention that comprises a dispersin and a protease,

(a) is a solid, preferably granular, laundry detergent composition and further comprises

(a1) at least one zeolite builder, preferably in an amount of 10 to 50 wt.-%, more preferably 20-30 wt.-%;

(a2) at least one phosphonate builder, preferably in an amount of 0.1 to 5 wt.-%, more preferably 0.4 to 1.5 wt.-%;

(a3) at least one further enzyme, preferably a cellulase, preferably in an amount of active enzyme of 100 to 5000 ppb, more preferably 1000 to 2000 ppb; and

(a4) at least one polymer, preferably a polyvinylpyrrolidon polymer, preferably in an amount of 0.01 to 1 wt.-%, more preferably 0.1 to 0.3 wt.-%; or

(b) is a solid laundry detergent composition and further comprises

(b1) at least one silicate builder, preferably in an amount of 2 to 20 wt.-%, more preferably 5-10 wt.-%;

(b2) optionally carboxymethylcellulose, preferably in an amount of 0.1 to 10 wt.-%, more preferably 0.1 to 4 wt.-%;

(b3) at least one further enzyme, preferably a cellulase, preferably in an amount of active enzyme of 0.1 to 100 ppm, more preferably 0.1 to 10 ppm;

(b4) optionally at least one soil release polymer, preferably a polyvinylpyrrolidon polymer, in an amount of 0.1 to 3 wt.-%, more preferably 0.1 to 1.0 wt.-%; and

(b5) at least one bleaching system, comprising a bleaching agent, a bleach activator and a bleach catalyst, preferably in an amount of 0.1 to 50 wt.-%, more preferably 0.1 to 30 wt.-%; or

(c) is liquid laundry detergent composition and further comprises

(c1) at least one surfactant, preferably nonionic surfactant, preferably in an amount of 1 to 20 wt.-%, preferably 3 to 15 wt.-%;

(c2) optionally at least one phosphonate builder, preferably in an amount of 0.1 to 3 wt.-%, more preferably 0.25 to 1.5 wt.-%

(c3) optionally at least at least one further enzyme, preferably a cellulase, preferably in an amount of enzyme composition of 0.001 to 1 wt.-%, more preferably 0.001 to 0.6 wt.-%; and

(c4) optionally at least one organic solvent, preferably glycerol, preferably in an amount of 0.1 to 10 wt.-%, more preferably 0.1 to 5 wt.-%; or

(d) is a liquid laundry detergent in unit dose form, preferably a pouch comprising a water-soluble film, and further comprises

(d1) water in an amount of up to 20 wt.-%, preferably 5 to 15 wt.-%;

(d2) optionally at least one bittering agent, preferably Benzyldiethyl(2,6-xylylcarbamoyl)-methylammoniumbenzoate, preferably in an amount of 0.00001 to 0.04 wt.-%;

(d3) optionally at least one optical brightener, preferably in an amount of 0.01 to 2 wt.-%, more preferably 0.01 to 1 wt.-%; and

(d4) optionally at least one polymer, preferably in an amount of 0.01 to 7 wt.-%, more preferably 0.1 to 5 wt.-%; or

(e) is a fabric finisher and further comprises

(e1) at least one softening silicone, preferably an amino-functionalized silicone, preferably in an amount of 0.1 to 10 wt.-%, more preferably 0.1 to 2 wt.-%;

(e2) at least one perfume, preferably at least partially encapsulated in microcapsules, more preferably at least partially encapsulated in melamine-formaldehyde microcapsules, preferably in an amount of 0.01 to 3 wt.-%, more preferably 0.1 to 1 wt.-%;

(e3) optionally polyquaternium 10 in an amount of 0.1 to 20 wt.-%, preferably 0.1 to 13 wt.-%;

(e4) optionally polyquaternium 37 in an amount of 0.1 to 20 wt.-%, preferably 0.1 to 13 wt.-%;

(e5) optionally a plant-based esterquat, preferably a canola- or palm-based esterquat, in an amount of 0.1 to 20 wt.-%, preferably 0.1 to 13 wt.-%; and

(e6) optionally adipic acid, in an amount of 0.1 to 20 wt.-%, preferably 0.1 to 13 wt.-%; or

(f) is an acidic cleaning agent, preferably having a pH less than 6, and further comprises

(f1) plant-based or bio-based surfactants, preferably each in an amount of 0.1 to 5, more preferably each in an amount of 0.1 to 2 wt.-%;

(f2) at least one acidic biocide, preferably selected from acids, more preferably HCl and formic acid; and

(f3) at least one soil release, water repellant or water spreading polymer, preferably in an amount of 0.01 to 3 wt.-%, more preferably 0.01 to 0.5 wt.-%; or

(g) is a neutral cleaning agent, preferably having a pH between 6.0 and 7.5, and further comprises

(g1) plant-based or bio-based surfactants, preferably each in an amount of 0.1 to 5, more preferably each in an amount of 0.1 to 2 wt.-%;

(g2) at least one biocide, preferably selected from quaternary ammonium compounds and alcohols; and

(g3) at least one soil release, water repellant or water spreading polymer, preferably in an amount of 0.01 to 3 wt.-%, more preferably 0.01 to 0.5 wt.-%; or

(h) is an alkaline cleaning agent, preferably having a pH of more than 7.5, and further comprises

(h1) plant-based or bio-based surfactants, preferably each in an amount of 0.1 to 5, more preferably each in an amount of 0.1 to 2 wt.-%; or

(i) is a hand dishwashing agent, preferably liquid hand dishwashing agent, and further comprises

(i1) at least one anionic surfactant, preferably in an amount of 0.1 to 40 wt.-%, more preferably 5 to 30 wt.-%;

(i2) at least one amphoteric surfactant, preferably betain, preferably in an amount of 0.1 to 25 wt.-%, more preferably 1 to 15 wt.-%;

(i3) at least one nonionic surfactant, preferably in an amount of 0.1 to 25 wt.-%, more preferably 2 to 10 wt.-%;

(i4) at least one further enzyme, preferably selected from amylases, preferably in an amount of enzyme composition of up to 1 wt.-%, more preferably up to 0.6 wt.-%; or

(j) is an automatic dishwashing composition and further comprises

(j1) at least one builder selected from citrate, aminocarboxylates and combinations thereof, preferably in an amount of 5 to 30 wt.-%, more preferably 10 to 20 wt.-%;

(j2) at least one phosphonate builder, preferably in an amount of 0.1 to 5 wt.-%, more preferably 0.4 to 1.5 wt.-%;

(j3) at least one nonionic surfactant, preferably in an amount of 0.1 to 10 wt.-%, more preferably 1 to 5 wt.-%;

(j4) at least one bleaching system, comprising a bleaching agent, a bleach activator and a bleach catalyst, preferably in an amount of 0.1 to 50 wt.-%, more preferably 0.1 to 30 wt.-%; and

(j5) at least one polymer selected from sulfopolymers, cationic polymers and polyacrylates, preferably in an amount of 0.01 to 15 wt.-%, more preferably 2 to 10 wt.-%; or

(k) further comprises

(k1) at least one sulfopolymer, preferably in an amount of 1 to 15, more preferably 2 to 10 wt.-% and is preferably a dishwashing, more preferably an automatic dishwashing composition; or

(l) further comprises at least one adjunct ingredient selected from probiotics, preferably microbes, spores or combinations thereof; or

(m) is in unit dose form and comprises at least 2, preferably 2, 3, 4 or 5 separate compartments; or

(n) is a phosphate-free composition.

When in the following reference is made to “(cleaning) compositions of the invention” or “(cleaning) compositions as described herein” or “(cleaning) compositions as defined herein”, in various embodiments the above-specified compositions (a)-(n) are meant. However, the invention is not limited to those compositions and is intended to cover other suitable cleaning compositions that comprise the enzyme mixtures defined herein. When in the following reference is made to a “protease” in the claimed combinations of a dispersin and a protease, the protease is one of the proteases that are defined herein. Furthermore, if not indicated otherwise, all references to percentages in relation to the disclosed compositions relate to wt % relative to the total weight of the respective composition. It is understood that when reference is made to compositions that contain enzymes as defined herein, the respective composition contains at least one of each of the specified enzymes but can also comprise two or more of each enzyme type, such as two or more dispersins and/or two or more proteases.

The invention further relates to the use of a composition for deep cleaning of an item, wherein the item is a textile or a surface. The invention further relates to use of a cleaning composition comprising a dispersin and a protease and, optionally, at least one cleaning component for deep cleaning of an item, wherein the item is a textile or a surface and wherein the protease is as defined herein.

The invention further relates to a method of formulating a cleaning composition comprising combining a dispersin, a protease and at least one cleaning component. The invention further relates to a kit intended for deep cleaning, wherein the kit comprises a solution of an enzyme mixture comprising a dispersin and a protease.

The invention further relates to a method of deep cleaning of an item, comprising the steps of: a) contacting the item with a solution comprising an enzyme mixture comprising a dispersin, a protease and a cleaning component, wherein the cleaning component is selected from 1 to 40 wt % of at least one a surfactant; 1 to 30 wt % of at least one builder; and 1 to 20 wt % of at least one bleach component; and b) optionally rinsing the item, wherein the item is preferably a textile.

The invention further relates to a method of deep cleaning of an item, comprising the steps of:
a) contacting the item with a cleaning composition comprising a dispersin and a protease and,
optionally, at least one cleaning component; and
b) optionally rinsing the item,
wherein the item is preferably a textile.

### DETAILED DESCRIPTION

Components of organic matter such as biofilm and EPS (extracellular polymeric substance) constitute a challenging type of staining due to the complex nature of such organic matters, and commercially available cleaning compositions are unable to effectively remove or reduce EPS and/or biofilm related stains. Biofilm may be produced when a group of microorganisms' cells stick to each other or stick to a surface, such as a textile, dishware or hard surface. These adherent cells are frequently embedded within a self-produced matrix of extracellular polymeric substance (EPS), which constitute 50% to 90% of the biofilm's total organic matter. EPS is mostly composed of polysaccharides (exopolysaccharides) and proteins, but include other macro-molecules such as eDNA, lipids and other organic substances. These proteins and polysaccharides such as PNAG are difficult to remove with the traditional cleaning compositions. Further, organic matter such EPS or biofilm may be sticky or gluing, which when present on textile may give rise to redeposition or backstaining of soil, resulting in a greying of the textile. When dirty laundry items are washed together with less dirty laundry items the dirt present in the wash liquor tends to stick to organic matter such as biofilm or biofilm components, and as a result the laundry item may be more “soiled” after wash than before wash, which is termed re-deposition. Another drawback of the presence organic matter e.g. biofilm is the malodor.

The compositions of the invention comprise a blend of dispersin and protease and effectively reduce or remove organic components, such as protein and PNAG, from surfaces such as textiles and hard surfaces e.g. dishes.

The compositions of the invention comprising a blend of dispersin and protease and effectively reduce or limit redeposition when applied in e.g. a laundry process.

The compositions of the invention comprising a blend of dispersin and protease and effectively reduce or limit malodor of e.g. textiles or hard surfaces such as dishes.

The compositions of the invention comprising a blend of dispersin and protease and improve whiteness of textiles.

A composition of the invention is a cleaning composition comprising at least one dispersin and at least one protease, as defined herein. Examples of useful dispersins are mentioned below in the section “Polypeptides having hexosaminidase activity”. The section “Polypeptides having protease activity” defines the proteases to be used in combination with the dispersins.

As shown in the example herein, it has been found that a combination of a dispersin and a protease is able to provide improved deep-cleaning of a textile compared to the individual enzymes. It is contemplated that this may be the result of the different EPS components targeted by these enzymes being localized in complex macromolecular structures which shield each other from enzymatic hydrolysis, and that use of the two different enzymes allows these complex structures to be degraded, thereby facilitating removal of the EPS biofilm.

Polypeptides Having Hexosaminidase Activity (Hexosaminidases)

The term hexosaminidase includes “dispersin” and the abbreviation “Dsp”, and means a polypeptide having hexosaminidase activity, EC 3.2.1., that catalyzes the hydrolysis of β-1,6-glycosidic linkages of N-acetyl-glucosamine polymers found e.g. in biofilm. The term hexosaminidase includes polypeptides having N-acetylglucosaminidase activity and β-N-acetylglucosaminidase activity. The term “polypeptide having hexosaminidase activity” may be used interchangeably with the term hexosaminidases and similarly the term “polypeptide having β-N-acetylglucosaminidase activity” may be used interchangeably with the term β-N-acetylglucosaminidases. For purposes of the present invention, hexosaminidase activity is determined according to the procedure described in Assay II. In a preferred embodiment, the polypeptide having hexosaminidase activity is a dispersin. In a preferred embodiment, the polypeptide having hexosaminidase activity is a β-N-acetylglucosaminidase targeting poly-β-1,6-N-acetylglucosamine.

In one embodiment, the invention relates to a composition comprising a protease, as defined herein, a hexosaminidase, preferably a β-N-acetylglucosaminidase e.g. a dispersin, and a cleaning component.

One embodiment of the invention relates to a composition comprising a hexosaminidase polypeptide, preferably a β-N-acetylglucosaminidase, e.g. a dispersin, wherein the polypeptide is selected from the group consisting of:
a) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 1,
b) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 2,
c) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 3,
d) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 4,
e) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 5,
f) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 6,
g) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 7,
h) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 8,
i) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 9,
j) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 10,
k) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 11,
l) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 12,
m) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 13,
n) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 14,
o) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 15,
p) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 16,
q) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 17,
r) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 18, and wherein the polypeptide cleaves β-substituted N-acetyl glucosaminide,
s) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 19, and wherein the polypeptide cleaves β-substituted N-acetyl glucosaminide,
t) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 20, and wherein the polypeptide cleaves β-substituted N-acetyl glucosaminide,
u) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 21, and wherein the polypeptide cleaves β-substituted N-acetyl glucosaminide,
v) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 22, and wherein the polypeptide cleaves β-substituted N-acetyl glucosaminide, and
w) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 23 and wherein the polypeptide cleaves β-substituted N-acetyl glucosaminide.

A polypeptide having hexosaminidase activity may be obtained from microorganisms of any genus. Preferably the hexosaminidase or the β-N-acetylglucosaminidase targeting poly-β-1,6-N-acetylglucosamine e.g. a dispersin, is obtained from Terribacillus, Curtobacterium, Aggregatibacter, Haemophilus, Actinobacillus, Lactobacillus or Staphylococcus, preferably Terribacillus or Lactobacillus. Alternatively, it may e.g. be obtained from Neisseria, Otariodibacter, Lactococcus, Frigoribacterium, Basfia, Weissella, Macrococcus or Leuconostoc.

In another embodiment, the polypeptide is an Aggregatibacter polypeptide, e.g., a polypeptide obtained from Aggregatibacter actinomycetemcomitans. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 1 and is obtained from Aggregatibacter, preferably Aggregatibacter actinomycetemcomitans.

In another embodiment, the polypeptide is a Haemophilus polypeptide, e.g., a polypeptide obtained from Haemophilus sputorum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2 and is obtained from Haemophilus, preferably Haemophilus sputorum.

In another embodiment, the polypeptide is an Actinobacillus polypeptide, e.g., a polypeptide obtained from Actinobacillus suis. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 3 and is obtained from Actinobacillus, preferably Actinobacillus suis.

In another embodiment, the polypeptide is an Actinobacillus polypeptide, e.g., a polypeptide obtained from Actinobacillus capsulatus DSM 19761. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 4 and is obtained from Actinobacillus, preferably Actinobacillus capsulatus DSM 19761.

In another embodiment, the polypeptide is an Actinobacillus polypeptide, e.g., a polypeptide obtained from Actinobacillus equuli subsp. equuli. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 5 and is obtained from Actinobacillus, preferably Actinobacillus equuli subsp. equuli.

In another embodiment, the polypeptide is an Aggregatibacter polypeptide, e.g., a polypeptide obtained from Aggregatibacter actinomycetemcomitans. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 6 and is obtained from Aggregatibacter, preferably Aggregatibacter actinomycetemcomitans.

In another embodiment, the polypeptide is an Aggregatibacter polypeptide, e.g., a polypeptide obtained from Aggregatibacter actinomycetemcomitans. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 7 and is obtained from Aggregatibacter, preferably Aggregatibacter actinomycetemcomitans.

In another embodiment, the polypeptide is an Actinobacillus polypeptide, e.g., a polypeptide obtained from Actinobacillus pleuropneumoniae. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 8 and is obtained from Actinobacillus, preferably Actinobacillus pleuropneumoniae.

In another embodiment, the polypeptide is a Curtobacterium polypeptide, e.g., a polypeptide obtained from Curtobacterium oceanosedimentum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 9 and is obtained from Curtobacterium, preferably Curtobacterium oceanosedimentum.

In another embodiment, the polypeptide is a Curtobacterium polypeptide, e.g., a polypeptide obtained from Curtobacterium flaccumfaciens. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 10 and is obtained from Curtobacterium, preferably Curtobacterium flaccumfaciens.

In another embodiment, the polypeptide is a Curtobacterium polypeptide, e.g., a polypeptide obtained from Curtobacterium luteum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 11 and is obtained from Curtobacterium, preferably Curtobacterium luteum.

In another embodiment, the polypeptide is a Curtobacterium polypeptide, e.g., a polypeptide obtained from Curtobacterium oceanosedimentum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 12 and is obtained from Curtobacterium, preferably Curtobacterium oceanosedimentum.

In another embodiment, the polypeptide is a Curtobacterium polypeptide, e.g., a polypeptide obtained from Curtobacterium leaf154. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 13 and is obtained from Curtobacterium, preferably Curtobacterium leaf154.

In another embodiment, the polypeptide having hexosaminidase activity is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 14 and is obtained from Terribacillus, preferably Terribacillus saccharophilus.

In another embodiment, the polypeptide is a Terribacillus polypeptide, e.g., a polypeptide obtained from Terribacillus goriensis. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 15 and is obtained from Terribacillus, preferably Terribacillus goriensis.

In another embodiment, the polypeptide is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 16 and is obtained from Terribacillus, preferably Terribacillus saccharophilus.

In another embodiment, the polypeptide is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 17 and is obtained from Terribacillus, preferably Terribacillus saccharophilus.

In another embodiment, the polypeptide is a Terribacillus polypeptide, e.g., a polypeptide obtained from Terribacillus saccharophilus. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 18 and is obtained from Terribacillus, preferably Terribacillus saccharophilus.

In another embodiment, the polypeptide is a Lactobacillus polypeptide, e.g., a polypeptide obtained from Lactobacillus paraplantarum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 19 and is obtained from Lactobacillus, preferably Lactobacillus paraplantarum.

In another embodiment, the polypeptide is a Lactobacillus polypeptide, e.g., a polypeptide obtained from Lactobacillus apinorum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 20 and is obtained from Lactobacillus, preferably Lactobacillus apinorum.

In another embodiment, the polypeptide is a Lactobacillus polypeptide, e.g., a polypeptide obtained from Lactobacillus paraplantarum. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 21 and is obtained from Lactobacillus, preferably Lactobacillus paraplantarum.

In another embodiment, the polypeptide is a Staphylococcus polypeptide, e.g., a polypeptide obtained from Staphylococcus cohnii. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 22 and is obtained from Staphylococcus, preferably Staphylococcus cohnii.

In another embodiment, the polypeptide is a Staphylococcus polypeptide, e.g., a polypeptide obtained from Staphylococcus fleurettii. In a preferred embodiment, the polypeptide is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 23 and is obtained from Staphylococcus, preferably Staphylococcus fleurettii.

The polypeptides useful in the present invention belong to the Glycoside Hydrolase family 20 (GH20, www.cazy.org). This family includes dispersins such as Dispersin B (DspB) which are β-N-acetylglucosaminidases belonging to the Glycoside Hydrolase 20 family.

The hexosaminidase can be included in the cleaning composition of the present invention at a level of from 0.01 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, from 250 ppm to 500 ppm.

The hexosaminidase can be included in the wash liquor solution at a level of from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, from 0.1 ppm to 10 ppm, from 0.2 ppm to 10 ppm, from 0.5 ppm to 5 ppm.

The dispersins above may be combined with any of the proteases below to form a blend to be added to a composition according to the invention. In various embodiments, the dispersin is a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 17.

Polypeptides Having Protease Activity

Generally, suitable proteases may be of any origin, but are preferably of bacterial or fungal origin, optionally in the form of protein engineered or chemically modified mutants. The protease may be an alkaline protease, such as a serine protease

According to the invention, the proteases useful in the present invention are selected from the group consisting of:

In one embodiment of the invention, the protease comprises an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99E in combination with at least two further amino acid substitutions selected from the group consisting of S3T, V4I and V199I, wherein positional numbering is according to SEQ ID NO:24. In another embodiment of the invention, the protease comprises an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99D in combination with at least two further amino acid substitutions selected from the group consisting of S3T, V4I and V199I, wherein positional numbering is according to SEQ ID NO:24.

Particularly Preferred are the Following Proteases:

Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99E in combination with amino acid substitutions S3T and V4I, preferably a protease according to SEQ ID NO:24 with the amino acid substitutions S3T, V4I and R99E.

Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99E in combination with amino acid substitutions S3T and V199I, preferably a protease according to SEQ ID NO:24 with the amino acid substitutions S3T, V199I and R99E.

Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99E in combination with amino acid substitutions V4I and V199I, preferably a protease according to SEQ ID NO:24 with the amino acid substitutions V4I, R99E and V199I.

Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99D in combination with amino acid substitutions S3T and V4I, preferably a protease according to SEQ ID NO:24 with the amino acid substitutions S3T, V4I and R99D.

Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99D in combination with amino acid substitutions S3T and V199I, preferably a protease according to SEQ ID NO:24 with the amino acid substitutions S3T, V199I and R99D.

Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99D in combination with amino acid substitutions V4I and V199I, preferably a protease according to SEQ ID NO:24 with the amino acid substitutions V4I, R99D and V199I.

Further particularly preferred proteases are characterized in that they comprise the amino acid substitution R99E or R99D in combination with all three amino acid substitutions S3T, V4I und V199I.

Accordingly, the following proteases are most preferred: Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99E in combination with amino acid substitutions S3T; V4I and V199I, preferably a protease according to SEQ ID NO:24 with the amino acid substitutions S3T, V4I, R99E, and V199I. Such a protease is set forth in SEQ ID NO:25.

Protease comprising an amino acid sequence having at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:24 over its entire length and comprises the amino acid substitution R99D in combination with amino acid substitutions S3T; V4I and V199I, preferably a protease according to SEQ ID NO:24 with the amino acid substitutions S3T, V4I, R99D, and V199I. Such a protease is set forth in SEQ ID NO:26.

Further preferred proteases are those as described above that additionally comprise the amino acid leucine (L) at position 211 in the positional numbering according to SEQ ID NO:24.

Alternatively or additionally, the protease may be characterized in that it is obtainable from a protease according to the invention by fragmentation, deletion, insertion or substitution mutagenesis and comprises an amino acid sequence that is over a length of at least 50, 60, 70, 80, 90, 100, 110, 120, 130, 140, 150, 160, 170, 180, 190, 200, 210, 220, 230, 240, 250, 260, 265 or 266 continuous amino acids identical to that of the starting molecule, wherein the amino acid substitutions according to the invention, i.e. R99E or R99D in combination with at least two of S3T, V4I and V199I, are invariable and remain part of the protease.

Alternatively or additionally, the protease may be characterized in that it is obtainable from a protease according to the invention by one or more amino acid substitutions in positions that correspond to positions 36, 42, 47, 56, 61, 69, 87, 96, 101, 102, 104, 114, 118, 120, 130, 139, 141, 142, 154, 157, 188, 193, 205, 211, 224, 229, 236, 237, 242, 243, 255 and 268 in SEQ ID NO:24, according to an alignment, wherein the amino acid substitutions according to the invention, i.e. R99E or R99D in combination with at least two of S3T, V4I and V199I, are invariable and remain part of the protease.

The alignment is typically based on the mature protease and also applies if the amino acid number of the protease of the invention is higher than that of the protease of SEQ ID NO:24.

Preferred positions for sequence alterations, in particular substitutions, of the protease derived from Bacillus lentus, which are of relevance in the corresponding homologous positions of the protease of the invention as they may impart advantageous properties to the protease, are therefore those positions that correspond to positions 36, 42, 47, 56, 61, 69, 87, 96, 101, 102, 104, 114, 118, 120, 130, 139, 141, 142, 154, 157, 188, 193, 205, 211, 224, 229, 236, 237, 242, 243, 255 and 268 of SEQ ID NO:24, as identified in a proper alignment. The wild type molecule comprises the following amino acid residues at those positions: S36, N42, A47, T56, G61, T69, E87, A96, A101, 1102, S104, N114, H118, A120, S130, S139, T141, S142, S154, S157, A188, V193, G205, L211, A224, K229, S236, N237, N242, H243, N255 and T268.

Preferred are, for example, substitutions 61A, 154D, 154E, A188P or V193M, insofar the corresponding positions are not naturally occupied by any of these preferred amino acids.

In various embodiments of the invention, all proteases described above, i.e. having various amino acid substitutions or other mutations or being fragments of those, still retain at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity relative to the amino acid sequence set forth in SEQ ID NO:24 over their entire length.

In one embodiment, the protease is protease having at least 90% sequence identity to SEQ ID NO:27, preferably at least 95% sequence identity, preferably at least 96% sequence identity, preferably at least 97% sequence identity, preferably at least 98% sequence identity to SEQ ID NO:27, wherein the protease variant has a glutamic acid residue (E) in position 101, and wherein the protease variant further comprises one or more substitutions selected from S156D; L262E; Q137H; S3T; R45E,D; P55N; T58W,Y,L; Q59D,M,N,T; G61 D,R; S87E; G97S; A98D,E,R; S106A,W; N117E; H120V,D,K,N; S124M; P129D; E136Q; S143W; S161T; S163A,G; Y171L; A172S; N185Q; V199M; Y209W; M222Q; N238H; V244T; N261T; L262N,Q,D. Said protease has increased stability in a liquid detergent compositions compared to the protease having the amino acid sequence of SEQ ID NO:27, and wherein the positions corresponds to the positions of SEQ ID NO:28.

In various embodiments, the protease comprises one or more substitutions selected from the group consisting of: R45E,D,Q; T58L; G61 D; S87E; G97S; A98E; S160A; N117E; H120D,K,V; P129D; E136Q; Q137H; S156D; S161T; S163A,G; V199M; M222Q; N261T and L262E,Q,N. Examples of preferred proteases of this embodiment include protease variants having at least 90% sequence identity to SEQ ID NO:27 and comprising one or more of the substitutions R45E,D,Q; Q59D; T58L; G61D; S87E; G97S; A98E; S106A; N117E; H120D,K,V; P129D; E136Q; Q137H; S156D; S161T; S163A,G; V199M; M222Q; N261T; L262E,Q,N.

The variants may have other substitutions e.g. substitutions known in the art to impart a particular beneficial property to the subtilase variants. There is an abundance of substitutions in subtilases known in the art and it is contemplated that such known substitutions may be used in the present invention in order to impart such known beneficial effects to the variants comprised in the detergent compositions of the present invention. The subtilase variants of the invention may comprise one or more further substitutions which may be used in the present invention in order to impart additional beneficial effects and/or to improve an existing effect such as stability and wash performance.

Preferred additional mutations includes one or more of the following substitutions V4I, N76D, V104T, N128Q, S141H, R145H, A194P, G195E, V205I, N218Q, A228V, N238E, or S265H.

Particular preferred examples of cleaning compositions according to the invention comprise subtilase variants having improved stability and/or improve wash performance in liquid detergent compared with the subtilase having the amino acid sequence of SEQ ID NO:27 and include variants comprising the amino acid sequences:

SEQ ID NO:27+S3T,

SEQ ID NO:27+R45E,D

SEQ ID NO:27+P55N,

SEQ ID NO:27+T58W,Y,L,

SEQ ID NO:27+Q59D,M,N,T,

SEQ ID NO:27+G61D,R,

SEQ ID NO:27+S87E,

SEQ ID NO:27+G97S,

SEQ ID NO:27+A98D,E,R,

SEQ ID NO:27+S106A,W,

SEQ ID NO:27+N117E,

SEQ ID NO:27+H120V,D,K,N,

SEQ ID NO:27+S124M,

SEQ ID NO:27+P129D

SEQ ID NO:27+E136Q,

SEQ ID NO:3+S143W,

SEQ ID NO:3+S161T,

SEQ ID NO:3+S163A,G,

SEQ ID NO:3+Y171L,

SEQ ID NO:27+A172S,

SEQ ID NO:27+N185Q,

SEQ ID NO:27+V199M,

SEQ ID NO:27+Y209W,

SEQ ID NO:27+M222Q,

SEQ ID NO:27+N238H,

SEQ ID NO:27+V244T,

SEQ ID NO:27+N261T,

SEQ ID NO:27+L262N,Q,D,E

SEQ ID NO:27+N76D+S163G+N238E

SEQ ID NO:27+S156D+L262E

SEQ ID NO:27+N238E+L262E

SEQ ID NO:27+S3T+N76D+S156D+Y209W

SEQ ID NO:27+H120D+S163G+N261D

SEQ ID NO:27+S163G+N128Q+N238E+L262E

SEQ ID NO:27+K27Q+H120D+S163G+N261D

SEQ ID NO:27+V104T+H120D+S156D+L262E

SEQ ID NO:27+G195E+V199M

SEQ ID NO:27+S3T+V4I+N261D

SEQ ID NO:27+A194P+G195E+V199M+V205I

SEQ ID NO:27+H120D+A228V

SEQ ID NO:27+S3T+V4I+A228V

SEQ ID NO:27+H120D+N261D

SEQ ID NO:27+H120D+S163G+N261D

SEQ ID NO:27+N76D+A228V+L262E

SEQ ID NO:27+N76D+Q137H+S141H+R145H+S163G+N238E

SEQ ID NO:27+Q137H+S141H+R145H+N238E+L262E

SEQ ID NO:27+S3T+N76D+Q137H+S141H+R145H+S156D+Y209W

SEQ ID NO:27+H120D+Q137H+S141H+R145H+S163G+N261D

SEQ ID NO:27+N76D+Q137H+S141H+R145H+A228V+N261D

SEQ ID NO:27+A194P+G195E+V199M+V205I+A228V+N261D

SEQ ID NO:27+N62D+H120D

SEQ ID NO:27+H120D+N261D

SEQ ID NO:27+N76D+N261D

SEQ ID NO:27+N76D+A228V+N261D

SEQ ID NO:27+A194P+G195E+V205I+N261D

SEQ ID NO:27+N76D+H120D+N261D

SEQ ID NO:27+H120D+S163G+N261D

SEQ ID NO:27+S3T+Q59D+N76D

SEQ ID NO:27+S3T+N76D+H120D

SEQ ID NO:27+S3T+N76D+A194P+G195E+V199M+V205I

SEQ ID NO:27+S3T+N76D+S156D

SEQ ID NO:27+S3T+N76D+Y209W+N261D

SEQ ID NO:27+S3T+N76D+H120D+Y209W

SEQ ID NO:27+S3T+N76D+S156D+Y209W

SEQ ID NO:27+S3T+V4I+N76D+A228V+N261D

SEQ ID NO:27+S3T+V4I+N76D+H120D

SEQ ID NO:27+H120D+P131F+A194P+N261D

SEQ ID NO:27+N76D+E136H+A228V+N261D

SEQ ID NO:27+N76D+N218S+A228V+N261D

SEQ ID NO:27+N76D+N218Q+A228V+N261D

SEQ ID NO:27+N76D+N218A+A228V+N261D

SEQ ID NO:27+K27Q+R45E

SEQ ID NO:27+N76D+A228V+L262E

SEQ ID NO:27+R45E+A88S

SEQ ID NO:27+S87E+K237E

SEQ ID NO:27+N261D+L262E

SEQ ID NO:27+S87E+L262E

SEQ ID NO:27+S87E+N238E

SEQ ID NO:27+K27Q+S87E

SEQ ID NO:27+N76D+N117E

SEQ ID NO:27+H120D+N238E

SEQ ID NO:27+Q59D+L262E

SEQ ID NO:27+K27Q+L262E

SEQ ID NO:27+H120D+L262E

SEQ ID NO:27+K27Q+Q59D

SEQ ID NO:27+K27Q+S156D

SEQ ID NO:27+K27Q+G61D

SEQ ID NO:27+Q59D+N261D

SEQ ID NO:27+Q59D+N117E

SEQ ID NO:27+K237E+N261D

SEQ ID NO:27+Q59D+N238E

SEQ ID NO:27+A15T+H120D+N261D

SEQ ID NO:27+N76D+S163G+N238E

SEQ ID NO:27+H120D+S163G+L262E

SEQ ID NO:27+H120D+S163G+N261D

SEQ ID NO:27+Q59D+H120D

SEQ ID NO:27+G61D+N76D

SEQ ID NO:27+S3T+N76D

SEQ ID NO:27+S3T+H120D

SEQ ID NO:27+G61D+H120D

SEQ ID NO:27+P55S+H120D

SEQ ID NO:27+S163G+A228V

SEQ ID NO:27+S163G+N261D

SEQ ID NO:27+S3T+S163G

SEQ ID NO:27+G61D+S163G

SEQ ID NO:27+S156D+S163G

SEQ ID NO:27+Q59D+S163G

SEQ ID NO:27+N76D+S163G

SEQ ID NO:27+P55S+S163G

SEQ ID NO:27+H120D+S163G

SEQ ID NO:27+T58L+Q59D

SEQ ID NO:27+P55S+T58L

SEQ ID NO:27+T58L+G97D

SEQ ID NO:27+T58L+S106A

SEQ ID NO:27+T58L+A228V

SEQ ID NO:27+S3T+T58L

SEQ ID NO:27+T58L+S156D

SEQ ID NO:27+T58L+Y91H

SEQ ID NO:27+T58L+H120D

SEQ ID NO:27+T58L+S163G

SEQ ID NO:27+S163G+N261D

SEQ ID NO:27+T58L+N261D

SEQ ID NO:27+T58L+N76D

SEQ ID NO:27+S3T+N76D+H120D

SEQ ID NO:27+S3T+N76D+A228V

SEQ ID NO:27+S3T+N76D+S156D

SEQ ID NO:27+S3T+N76D+Y209W

SEQ ID NO:27+S3T+N76D+Y209W+V244T

SEQ ID NO:27+N76D+H120D

SEQ ID NO:27+N76D+S156D

SEQ ID NO:27+H120D S156D

SEQ ID NO:27+R45E+L262E

SEQ ID NO:27+Q59D+G61D

SEQ ID NO:27+S87E+L262E

SEQ ID NO:27+G61D+L262E

SEQ ID NO:27+Q59D+L262E

SEQ ID NO:27+R45E+Q59D

SEQ ID NO:27+Q59D+S156D

SEQ ID NO:27+S156D+L262E

SEQ ID NO:27+S163G+N238E+L262E

SEQ ID NO:27+S3T+V4I+S163G+N261D

SEQ ID NO:27+H120D+S163G+N261D

SEQ ID NO:27+Y91H+N117H+N238H

SEQ ID NO:27+T58L+S163G+N261D

SEQ ID NO:27+S3T+V4I+S163G+N261D

SEQ ID NO:27+S87E+S163G+L262E

SEQ ID NO:27+S156D+S163G+L262E

SEQ ID NO:27+T58LS163G+N261D

SEQ ID NO:27+S156DS163G+L262E

SEQ ID NO:27+S3T+N76D+Y209W+N261 D+L262E

As examples of preferred variants having improved stability in liquid detergent and improved wash performance compared with the parent enzyme can be mentioned:

SEQ ID NO:27+R45E,D,Q

SEQ ID NO:27+Q58L

SEQ ID NO:27+Q59D,

SEQ ID NO:27+G61D,

SEQ ID NO:27+S87E,

SEQ ID NO:27+G97S,

SEQ ID NO:27+A98E,

SEQ ID NO:27+N117E,

SEQ ID NO:27+H120D,K,V

SEQ ID NO:27+P129D,

SEQ ID NO:27+E136Q,

SEQ ID NO:27+Q137H,

SEQ ID NO:27+S156D,

SEQ ID NO:27+S160A,

SEQ ID NO:27+S163A,G,

SEQ ID NO:27+V199M

SEQ ID NO:27+M222Q

SEQ ID NO:27+N261T

SEQ ID NO:27+L262E,Q,N.

These preferred variants have improved stability such as detergent stability and/or improved or on par wash performance compared with the parent subtilase. In this connection improved wash performance is intended to mean that the wash performance of the variant is higher on at least one stain than the wash performance of the parent subtilase where wash performance is determined using a suitable wash performance assay in a given detergent composition under suitable conditions.

The subtilase variants may further comprise one or more additional alterations at one or more (e.g., several) other positions, selected from the group consisting of positions: 3, 4, 9, 12, 14, 15, 40, 43, 68, 72, 79, 86, 88, 92, 98, 99, 101, 120, 146, 183, 184, 188, 194, 216, 218, 224, 228, 236, 245, 255, 261, 267 and 270, preferably positions 9, 15, 68 and/or 120 (numbering according to SEQ ID NO: 29). It will be clear to the skilled artisan that if a position has already been altered once, then it will not be altered a second time. In a preferred embodiment, the alteration at any of the positions selected from the group consisting of 3, 4, 9, 12, 14, 15, 40, 43, 68, 72, 79, 86, 88, 92, 98, 99, 101, 120, 146, 183, 184, 188, 194, 216, 218, 224, 228, 236, 245, 255, 261, 267 and 270 is a substitution. In a more preferred embodiment, the subtilase variant further comprises one or more substitutions selected from the group consisting of 3{D, E, L}, 4I, 9{H, K, R, G}, 12{D, E}, 14T, 15{G, M, S, T}, 40{A, G, M, S, T}, 43{D, E}, 63G, 68{A, G, I, L, M, S, T}, 72{V, L}, N76{D, E}, 79T, 86H, 88V, 92S, 98T, 99{E, T, A, G, M, D}, 101L, 120 {I, N}, 146S, 183{E, D}, 184{E, D}, 188G, 194P, 216{D, E}, 218{E, D}, 224{S, A, T, G, M}, 228T, 236D, 245{H, K, R}, 255{D, E}, 261{D, E}, 267{l, L, V} and/or 270{G, M, S, T} (numbering according to SEQ ID NO: 29). In an even more preferred embodiment, the subtilase variant further comprises one or more substitutions selected from the group consisting of S3{D, E, L}, V4I, S9{H, K, R, G}, Q12{D, E}, P14T, A15{G, M, S, T}, P40{A, G, M, S, T}, N43{D, E}, V68{A, G, I, L, M, S, T}, 172{V, L}, N76{D, E}, I79T, P86H, A88V, A92S, A98T, S99{E, T, A, G, M, D}, S101L, H120{I, N}, G146S, N183{E, D}, N184{E, D}, S188G, A194P, S216{D, E}, N218{E, D}, T224{S, A, T, G, M}, A228T, S236D, Q245{H, K, R}, T255{D, E}, N261{D, E}, L267{I, L, V} and/or A270{G, M, S, T} in the mature polypeptide of SEQ ID NO: 27, or a polypeptide having at least 60%, preferably at least 70%, preferably at least 80%, preferably at least 90%, preferably at least 95% sequence identity hereto, wherein each position corresponds to the corresponding position of the mature polypeptide of SEQ ID NO:28.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis. In the latter technique, single alanine mutations are introduced at every residue in the molecule, and the resultant mutant molecules are tested for protease activity to identify amino acid residues that are critical to the activity of the molecule. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. For BPN′ (SEQ ID NO:28) the catalytic triad comprising the amino acids S221, H64, and D32 is essential for protease activity of the enzyme.

The subtilase variants may consist of 150 to 350, e.g., 175 to 330, 200 to 310, 220 to 300, 240 to 290, 260 to 280 or 269, 270, 271, 272, 273, 274 or 275 amino acids.

According to one embodiment and/or according to any of the embodiments above the invention relates to a detergent composition comprising subtilase variant having at least 90% sequence identity to SEQ ID NO: 27, wherein the variant has a glutamic acid residue (E) in position corresponding to position 101 of SEQ ID NO:28, and where the variant has reduced cellulose binding compared to the subtilase having the amino acid sequence of SEQ ID NO:27. One embodiment relates to a detergent composition comprising subtilase variant having at least 90% sequence identity to SEQ ID NO:27, wherein the variant has a glutamic acid residue (E) in position corresponding to position 101 of SEQ ID NO:28, and where the variant has reduced cellulose binding compared to the subtilase having the amino acid sequence of SEQ ID NO:27 and wherein the variant comprises a substitution of a positively charged amino acid residue on the surface of the protease with a neutral or negatively charged residue; or a neutral residue on the surface of the protease is substituted with a negatively charged residue. According to one embodiment or any of the above embodiments the invention relates to a detergent composition comprising subtilase variant having at least 90% sequence identity to SEQ ID NO:27, wherein the variant has a glutamic acid residue (E) in position corresponding to position 101 of SEQ ID NO:28, and where the variant has reduced cellulose binding compared to the subtilase having the amino acid sequence of SEQ ID NO:27 and/or wherein the variant comprises a substitution of a positively charged amino acid residue on the surface of the protease with a neutral or negatively charged residue; or a neutral residue on the surface of the protease is substituted with a negatively charged residue wherein the subtilase variant comprising a substitution selected from the group consisting of V4D,E,I, R10N,Q,D,E,S, H17D, K27S,N,Q,E,D, N43E, 144V, R45E,D,Q,N, G46D, S49N,D, P52E, G53D,E, Q59D, G61D, N62D, L75D, N76D, 179D, S87E, G97D, A98E, *103aE, I104T, N117E, H120D, E136K,Q, S156D, R170E,Q,N,D,S N185D, G195E, N218A, K235L,W,N,Q,E,S, K237N,Q,D,E,S, N238D,E, V244D, R246Q,E,D, R247S,E, Q, D, K251S,D,Q,E,N, N261D, L262D,E and S265H, preferable the substitutions are selected among N117E, S156D, N238E, N261D and L262E and preferably the variant further comprise a substitution selected among: S3T, N128Q, Q137H, S141H, R145H, S163G, A194P, V199M, V205I, N218Q or A228V.

According to one embodiment and/or according to any of the above embodiment the invention relates to a detergent composition comprising subtilase variant having at least 90% sequence identity to SEQ ID NO:27, wherein the variant has a glutamic acid residue (E) in position corresponding to position 101 of SEQ ID NO:28, where the variant has reduced cellulose binding compared to the subtilase having the amino acid sequence of SEQ ID NO:27 and wherein the subtilase variant further comprising a substitutions selected among N117E+S3T, S156D+S3T, N238E+S3T, N261 D+S3T, L262E+S3T, N117E+N128Q, S156D+N128Q, N238E+N128Q, N261D+N128Q, L262E+N128Q, N117E+Q137H, S156D+Q137H, N238E+Q137H, N261 D+Q137H, L262E+Q137H, N117E+S141H, S156D+S141H, N238E+S141H, N261D+S141H, L262E+S141H, N117E+R145H, S156D+R145H, N238E+R145H, N261D+R145H, L262E+R145H, N117E+S163G, S156D+S163G, N238E+S163G, N261D+S163G, L262E+S163G, N117E+A194P, S156D+A194P, N238E+A194P, N261D+A194P, L262E+A194P, N117E+V199M, S156D+V199M, N238E+V199M, N261D+V199M, L262E+V199M, N117E+V205I, S156D+V205I, N238E+V205I, N261 D+V205I, L262E+V205I, N117E+N218Q, S156D+N218Q, N238E+N218Q, N261D+N218Q, L262E+N218Q, N117E+A228V, S156D+A228V, N238E+A228V, N261D+A228V, L262E+A228V.

According to one embodiment and/or according to any of the above embodiment the invention relates to a detergent composition comprising subtilase variant having at least 90% sequence identity to SEQ ID NO:27, wherein the variant has a glutamic acid residue (E) in position corresponding to position 101 of SEQ ID NO:28, where the variant has reduced cellulose binding compared to the subtilase having the amino acid sequence of SEQ ID NO:27 and wherein the subtilase variant further comprising a substitutions selected among:

SEQ ID NO:27+V4D,E,I

SEQ ID NO:27+R10N,Q,D,E,S,

SEQ ID NO:27+H17D,

SEQ ID NO:27+K27S,N,Q,E,D,

SEQ ID NO:27+R45E,D,Q,N,

SEQ ID NO:27+G53D,

SEQ ID NO:27+Q59D,

SEQ ID NO:27+G61D,

SEQ ID NO:27+L75D,

SEQ ID NO:27+N76D,

SEQ ID NO:27+I79D,

SEQ ID NO:27+S87E,

SEQ ID NO:27+G97D,

SEQ ID NO:27+A98E,

SEQ ID NO:27+*103aE,

SEQ ID NO:27+N117E,

SEQ ID NO:27+H120D,

SEQ ID NO:27+E136K,Q,

SEQ ID NO:27+S156D,

SEQ ID NO:27+R170E,Q,N,D,

SEQ ID NO:27+N185D,

SEQ ID NO:27+G195E,

SEQ ID NO:27+K235L,W,N,Q,E,S,

SEQ ID NO:27+K237N,Q,D,E,S,

SEQ ID NO:27+N238D,E,

SEQ ID NO:27+V244D

SEQ ID NO:27+R246Q,E,D,

SEQ ID NO:27+R247S,E,

SEQ ID NO:27+K251S,D,Q,E,N,

SEQ ID NO:27+N261D,

SEQ ID NO:27+L262D,E

SEQ ID NO:27+S265H

SEQ ID NO:27+A194P+G195E

SEQ ID NO:27+G195E+V199M

SEQ ID NO:27+N76D+A228V+N261D;

SEQ ID NO:27+N76D+S163G+N238E

SEQ ID NO:27+S156D+L262E

SEQ ID NO:27+N238E+L262E

SEQ ID NO:27+S3T+N76D+S156D+Y209W

SEQ ID NO:27+K27Q+H120D+S163G+N261D

SEQ ID NO:27+V104T+H120D+S156D+L262E

SEQ ID NO:27+V104T+S156D+L262E

SEQ ID NO:27+Q137H+S141H+R145H+N238E+L262E

SEQ ID NO:27+S3T+V4I+A228V;

SEQ ID NO:27+H120D S163G N261D

SEQ ID NO:27+N76D+S101E+A228V+L262E;

SEQ ID NO:27+N76D+Q137H+S141H+R145H+S163G+N238E

SEQ ID NO:27+S3T+N76D+Q137H+S141H+R145H+S156D+Y209W

SEQ ID NO:27+H120D+Q137H+S141H+R145H+S163G+N261D

SEQ ID NO:27+A194P+G195E+V199M+V205I;

SEQ ID NO:27+S3T+N76D+A194P+G195E+V199M+V205I;

SEQ ID NO:27+A228V+N261 D;

SEQ ID NO:27+N76D+A228V;

SEQ ID NO:27+S3T+V4I+N261D;

SEQ ID NO:27+H120D+A228V;

SEQ ID NO:27+N76D+N261D;

SEQ ID NO:27+A194P+G195E+V199M+V205I+A228V+N261 D;

SEQ ID NO:27+A194P+G195E+V205I+A228V; or

SEQ ID NO:27+H120D+N261D.

Preferably the variants are selected from the group consisting of:

SEQ ID NO:27+N238E+L262E

SEQ ID NO:27+S156D+L262E

SEQ ID NO:27+S3T+V4I+A228V;

SEQ ID NO:27+G195E+V199M

SEQ ID NO:27+H120D S163G N261D

SEQ ID NO:27+N76D+A228V+N261D;

SEQ ID NO:27+S3T+N76D+S156D+Y209W

SEQ ID NO:27+Q137H+S141H+R145H+N238E+L262E

SEQ ID NO:27+Q137H+S141H+R145H+S156D+L262E

SEQ ID NO:27+N76D+Q137H+S141H+R145H+A228V+N261D;

SEQ ID NO:27+N76D+Q137H+S141H+R145H+S163G+N238E

SEQ ID NO:27+H120D+Q137H+S141H+R145H+S163G+N261D

SEQ ID NO:27+S3T+N76D+Q137H+S141H+R145H+S156D+Y209W, wherein the positions correspond to the positions in SEQ ID NO:28 and wherein the subtilase variant has at least at least 60%, such as at least 70%, such as at least 80%, such as at least 90%, such as at least 95%, such as at least 98% or such as at least 99% sequence identity to SEQ ID NO:27.

In another embodiment, the protease is a protease comprising an amino acid sequence that has at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 98.8% sequence identity to the amino acid sequence set forth in SEQ ID NO:30 over its entire length and comprises an amino acid substitution in at least one position corresponding to positions 12, 43, 122, 127, 154, 156, 160, 211, 212 und 222 of SEQ ID NO:30.

In various embodiments, the amino acid substitutions are selected from the group consisting of 12L, 43V, 122L, 127P, 154S, 156A, 160S, 211N, 211L, 212D, 212H and 222S, in particular Q12L, 143V, M122L, D127P, N154S, T156A, G160S, M211N, M211L, P212D, P212H and A222S.

In various embodiments, the protease comprises at least one of the following sets of amino acid substitutions (1) 143V; (2) M122L, N154S and T156A; (3) M211N and P212D; (4) M211L and P212D; (5) G160S; (6) D127P, M211L und P212D; (7) P212H; or (8) Q12L, M122L und A222S, wherein the numbering is according to SEQ ID NO:30.

The amino acid sequences of such exemplary proteases are set forth in SEQ ID Nos: 31-38.

In another embodiment, the protease is a protease comprising an amino acid sequence that has at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4%, or 99.5% sequence identity to the amino acid sequence set forth in SEQ ID NO:39 over its entire length and comprising (i) at least two of the amino acid substitutions 3T, 4I, 99E and 199I at the positions corresponding to positions 3, 4, 99 and 199 of SEQ ID NO:39 and (ii) in at least one position corresponding to positions 74, 136, 143, 154, 161, 163, 171, 200, 203, 209, 212 or 256 of SEQ ID NO:39 at least one amino acid substitution, preferably at least one amino acid substitution selected from 74D, 74E, 74Q, 136Q, 143L, 143W, 143Y, 154D, 154Q, 161T, 163G, 171L, 200A, 200L, 200S, 200T, 203K, 203V, 203W, 209W, 212N, 212S, 212T, 256D, 256E or 256Q. Such proteases have improved stability and/or performance relative to the wild type (SEQ ID NO:39) or other variants and are thus particularly suited for use in cleaning compositions.

In various embodiments, the protease is a protease comprising an amino acid sequence that has at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4%, or 99.5% sequence identity to the amino acid sequence set forth in SEQ ID NO:39 over its entire length and comprising (i) at least three of the amino acid substitutions 3T, 4I, 99E and 199I at the positions corresponding to positions 3, 4, 99 and 199 of SEQ ID NO:39 and (ii) in at least one position corresponding to positions 74, 136, 143, 154, 161, 163, 171, 200, 203, 209, 212 or 256 of SEQ ID NO:39 at least one amino acid substitution, preferably at least one amino acid substitution selected from 74D, 74E, 74Q, 136Q, 143L, 143W, 143Y, 154D, 154Q, 161T, 163G, 171L, 200A, 200L, 200S, 200T, 203K, 203V, 203W, 209W, 212N, 212S, 212T, 256D, 256E or 256Q.

In various embodiments, the protease is a protease comprising an amino acid sequence that has at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4%, or 99.5% sequence identity to the amino acid sequence set forth in SEQ ID NO:39 over its entire length and comprising (i) the amino acid substitutions 3T, 4I, 99E and 199I at the positions corresponding to positions 3, 4, 99 and 199 of SEQ ID NO:39 and (ii) in at least one position corresponding to positions 74, 136, 143, 154, 161, 163, 171, 200, 203, 209, 212 or 256 of SEQ ID NO:39 at least one amino acid substitution, preferably at least one amino acid substitution selected from 74D, 74E, 74Q, 136Q, 143L, 143W, 143Y, 154D, 154Q, 161T, 163G, 171L, 200A, 200L, 200S, 200T, 203K, 203V, 203W, 209W, 212N, 212S, 212T, 256D, 256E or 256Q.

In the described positions the wild type protease (SEQ ID NO:39) comprises the amino acid residues: N74, A136, R143, S154, Y161, A163, V171, Q200, Y203, A209, N212, L256.

Exemplary variants comprise the following sets of substitutions: (i) S3T+V4I+R99E+V199I+Q200L+Y203W; (ii) S3T+V4I+R99E+V199I+N212S; (iii) S3T+V4I+R99E+V199I+N74D; (iv) S3T+V4I+R99E+V199I+S154D+L256E; (v) S3T+V4I+R99E+V199I+Q200L+Y203W+S154D+L256E; (vi) S3T+V4I+R99E+V199I+N74D+Q200L+Y203W; (vii) S3T+V4I+R99E+V199I+N74D+S154D+Q200L+Y203W+L256E; (viii) S3T+V4I+R99E+V199I+N74D+N212S; (ix) S3T+V4I+R99E+V199I+N74D+S154D+Y203W+L256E; (x) S3T+V4I+R99E+V199I+N74D+Y203W; (xi) S3T+V4I+R99E+V199I+N74D+S154D+Q200L+L256E; (xii) S3T+V4I+R99E+V199I+N74D+Q200L; (xiii) S3T+V4I+R99E+V199I+S154D+Q200L+Y203W; (xiv) S3T+V4I+R99E+V199I+Q200L+Y203W+L256E; (xv) S3T+V4I+R99E+V199I+A136Q+R143W+Y161T+Q200L; (xvi) S3T+V4I+R99E+V199I+N74D+R143Y+A209W+N212S+L256E; (xvii) S3T+V4I+R99E+V199I+A136Q+S154D+V171 L+Q200L, with the numbering according to SEQ ID NO:39.

Compositions

The invention relates to cleaning compositions, as defined herein, comprising a dispersin and a protease as per the appended claims.

One embodiment of the invention relates to a cleaning composition, as defined herein, comprising a dispersin, a protease and, optionally, at least one cleaning component. The protease is any of the proteases mentioned under the heading “Polypeptides having protease activity”.

It is contemplated that the proteases described herein may act synergistically with the dispersin in reduction and removal of biofilm or components hereof. Biofilm is a complex structure comprising e.g. protein and PNAG, where the target substrate e.g. the PNAG may be embedded in the biofilm structure, and it is believed that when the dispersins and proteases are acting together on respective parts of the biofilm structure, the PNAG and protein components may be more effectively removed. It is thus advantageous to formulate dispersins with proteases in cleaning compositions e.g. for deep cleaning.

One aspect of the invention thus relates to a method of formulating a cleaning composition comprising combining a dispersin, a protease, as defined herein, and at least one cleaning component. The invention further relates to a kit intended for deep cleaning, wherein the kit comprises a solution of an enzyme mixture comprising a dispersin and a protease, as defined herein.

Thus, as mentioned the protease should be compatible with cleaning components and likewise, the dispersins to be formulated together with the protease or to be used together with the protease should also be compatible with cleaning components. Dispersins are at present not standard ingredients in cleaning compositions. However, the applicant has identified dispersins suitable for use in cleaning compositions have been identified, e.g. in WO 2017/186936, WO 2017/186937 and WO 2017/186943. Enzymes such as dispersins should not only be compatible with the cleaning components, the dispersins should also be compatible with other enzymes which may be present in a typical cleaning composition. It is well known that proteases may negatively influence the performance of other enzymes as the protease may degrade these enzymes (being themselves proteins). Surprisingly, it has been found that proteases and dispersins not only are compatible but can even act synergistically in respect of biofilm stain reduction and removal e.g. in deep cleaning.

Particularly useful dispersins may be those of microbial origin. One embodiment of the invention relates to a cleaning composition, as defined herein, comprising a dispersin and a protease, wherein the dispersin is microbial, preferably obtained from bacteria or fungi. In one embodiment, the cleaning composition, as defined herein, comprises a dispersin from bacteria. One embodiment of the invention relates to a cleaning composition, as defined herein, comprising a dispersin and a protease, wherein the dispersin is obtained from Terribacillus, Curtobacterium, Aggregatibacter, Haemophilus, Actinobacillus, Lactobacillus or Staphylococcus preferably Terribacillus or Lactobacillus.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 2.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 3.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 4.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 5.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 6.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 7.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 8.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 9.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 10.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 11.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 12.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 13.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 14.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 15.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 16.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 17.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 18.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 19.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 20.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 21.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 22.

One embodiment of the invention relates to a cleaning composition, as defined herein, comprising a dispersin and a protease, wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 23.

One embodiment relates to a cleaning composition, as defined herein, comprising a Terribacillus dispersin and a protease, wherein the protease is any one of those described above under the section “Polypeptides having protease activity”.

One embodiment relates to a cleaning composition, as defined herein, comprising a Curtobacterium dispersin and a protease, wherein the protease is any one of those described above under the section “Polypeptides having protease activity”.

One embodiment relates to a cleaning composition, as defined herein, comprising a Aggregatibacter dispersin and a protease, wherein the protease is any one of those described above under the section “Polypeptides having protease activity”.

One embodiment relates to a cleaning composition, as defined herein, comprising a Haemophilus dispersin and a protease, wherein the protease is any one of those described above under the section “Polypeptides having protease activity”.

One embodiment relates to a cleaning composition, as defined herein, comprising a Actinobacillus dispersin and a protease, wherein the protease is any one of those described above under the section “Polypeptides having protease activity”.

One embodiment relates to a cleaning composition, as defined herein, comprising a Lactobacillus dispersin and a protease, wherein the protease is any one of those described above under the section “Polypeptides having protease activity”.

One embodiment relates to a cleaning composition, as defined herein, comprising a Staphylococcus dispersin and a protease, wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1. One embodiment relates to a cleaning composition, as defined herein, comprising a dispersin and a protease, wherein the dispersin comprises or consists of the polypeptide comprising the amino acid sequence shown in SEQ ID NO:1 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:2. One embodiment relates to a cleaning composition, as defined herein, comprising a dispersin and a protease, wherein the dispersin comprises or consists of the polypeptide comprising the amino acid sequence shown in SEQ ID NO:2 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 3. One embodiment relates to a cleaning composition, as defined herein, comprising a dispersin and a protease, wherein the dispersin comprises or consists of the polypeptide comprising the amino acid sequence shown in SEQ ID NO:3 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:4. One embodiment relates to a cleaning composition, as defined herein, comprising a dispersin and a protease, wherein the dispersin comprises or consists of the polypeptide comprising the amino acid sequence shown in SEQ ID NO:4 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:5. One embodiment relates to a cleaning composition, as defined herein, comprising a dispersin and a protease, wherein the dispersin comprises or consists of the polypeptide comprising the amino acid sequence shown in SEQ ID NO:5 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:6. One embodiment relates to a cleaning composition, as defined herein, comprising a dispersin and a protease, wherein the dispersin comprises or consists of the polypeptide comprising the amino acid sequence shown in SEQ ID NO:6 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:7. One embodiment relates to a cleaning composition, as defined herein, comprising a dispersin and a protease, wherein the dispersin comprises or consists of the polypeptide comprising the amino acid sequence shown in SEQ ID NO:7 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:8. One embodiment relates to a cleaning composition, as defined herein, comprising a dispersin and a protease, wherein the dispersin comprises or consists of the polypeptide comprising the amino acid sequence shown in SEQ ID NO:8 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:9. One embodiment relates to a cleaning composition, as defined herein, comprising a dispersin and a protease, wherein the dispersin comprises or consists of the polypeptide comprising the amino acid sequence shown in SEQ ID NO:9 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:10. One embodiment relates to a cleaning composition, as defined herein, comprising a dispersin and a protease, wherein the dispersin comprises or consists of the polypeptide comprising the amino acid sequence shown in SEQ ID NO:10 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:11. One embodiment relates to a cleaning composition, as defined herein, comprising a dispersin and a protease, wherein the dispersin comprises or consists of the polypeptide comprising the amino acid sequence shown in SEQ ID NO:11 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:12. One embodiment relates to a cleaning composition, as defined herein, comprising a dispersin and a protease, wherein the dispersin comprises or consists of the polypeptide comprising the amino acid sequence shown in SEQ ID NO:12 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:13. One embodiment relates to a cleaning composition, as defined herein, comprising a dispersin and a protease, wherein the dispersin comprises or consists of the polypeptide comprising the amino acid sequence shown in SEQ ID NO:13 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:14. One embodiment relates to a cleaning composition, as defined herein, comprising a dispersin and a protease, wherein the dispersin comprises or consists of the polypeptide comprising the amino acid sequence shown in SEQ ID NO:14 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:15. One embodiment relates to a cleaning composition, as defined herein, comprising a dispersin and a protease, wherein the dispersin comprises or consists of the polypeptide comprising the amino acid sequence shown in SEQ ID NO:15 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:16. One embodiment relates to a cleaning composition, as defined herein, comprising a dispersin and a protease, wherein the dispersin comprises or consists of the polypeptide comprising the amino acid sequence shown in SEQ ID NO:16 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 17. One embodiment relates to a cleaning composition, as defined herein, comprising a dispersin and a protease, wherein the dispersin comprises or consists of the polypeptide comprising the amino acid sequence shown in SEQ ID NO:17 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:18. One embodiment relates to a cleaning composition, as defined herein, comprising a dispersin and a protease, wherein the dispersin comprises or consists of the polypeptide comprising the amino acid sequence shown in SEQ ID NO:18 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:19. One embodiment relates to a cleaning composition, as defined herein, comprising a dispersin and a protease, wherein the dispersin comprises or consists of the polypeptide comprising the amino acid sequence shown in SEQ ID NO:19 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:20. One embodiment relates to a cleaning composition, as defined herein, comprising a dispersin and a protease, wherein the dispersin comprises or consists of the polypeptide comprising the amino acid sequence shown in SEQ ID NO:20 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:21. One embodiment relates to a cleaning composition, as defined herein, comprising a dispersin and a protease, wherein the dispersin comprises or consists of the polypeptide comprising the amino acid sequence shown in SEQ ID NO:21 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:22. One embodiment relates to a cleaning composition, as defined herein, comprising a dispersin and a protease, wherein the dispersin comprises or consists of the polypeptide comprising the amino acid sequence shown in SEQ ID NO:22 and wherein the protease is any one of those described above under the section “Polypeptides having protease activity”.

One embodiment relates to a cleaning composition, as defined herein, comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:23. One embodiment relates to a cleaning composition, as defined herein, comprising a dispersin and a protease, wherein the dispersin comprises or consists of the polypeptide comprising the amino acid sequence shown in SEQ ID NO:23 and wherein the protease is any one of those described above under the section “Polypeptides having protease activity”.

One embodiment of the invention relates to a cleaning composition, as defined herein, comprising a protease as described above under the section “Polypeptides having protease activity” and a polypeptide having dispersin activity, wherein the polypeptide is selected from the group consisting of:
a) a polypeptide having at least 60%, at least 65%, at least 70%, at least 75% at least 80%, at least 85%, at least 90%, at least 95%, at least 98% or 100% sequence identity to the polypeptide shown in SEQ ID NO: 1,
b) a polypeptide having at least 60%, at least 65%, at least 70%, at least 75% at least 80%, at least 85%, at least 90%, at least 95%, at least 98% or 100% sequence identity to the polypeptide shown in SEQ ID NO: 2,
c) a polypeptide having at least 60%, at least 65%, at least 70%, at least 75% at least 80%, at least 85%, at least 90%, at least 95%, at least 98% or 100% sequence identity to the polypeptide shown in SEQ ID NO: 3,
d) a polypeptide having at least 60%, at least 65%, at least 70%, at least 75% at least 80%, at least 85%, at least 90%, at least 95%, at least 98% or 100% sequence identity to the polypeptide shown in SEQ ID NO: 4,
e) a polypeptide having at least 60%, at least 65%, at least 70%, at least 75% at least 80%, at least 85%, at least 90%, at least 95%, at least 98% or 100% sequence identity to the polypeptide shown in SEQ ID NO: 5,
f) a polypeptide having at least 60%, at least 65%, at least 70%, at least 75% at least 80%, at least 85%, at least 90%, at least 95%, at least 98% or 100% sequence identity to the polypeptide shown in SEQ ID NO: 6,
g) a polypeptide having at least 60%, at least 65%, at least 70%, at least 75% at least 80%, at least 85%, at least 90%, at least 95%, at least 98% or 100% sequence identity to the polypeptide shown in SEQ ID NO: 7,
h) a polypeptide having at least 60%, at least 65%, at least 70%, at least 75% at least 80%, at least 85%, at least 90%, at least 95%, at least 98% or 100% sequence identity to the polypeptide shown in SEQ ID NO: 8,
i) a polypeptide having at least 60%, at least 65%, at least 70%, at least 75% at least 80%, at least 85%, at least 90%, at least 95%, at least 98% or 100% sequence identity to the polypeptide shown in SEQ ID NO: 9,
j) a polypeptide having at least 60%, at least 65%, at least 70%, at least 75% at least 80%, at least 85%, at least 90%, at least 95%, at least 98% or 100% sequence identity to the polypeptide shown in SEQ ID NO: 10,
k) a polypeptide having at least 60%, at least 65%, at least 70%, at least 75% at least 80%, at least 85%, at least 90%, at least 95%, at least 98% or 100% sequence identity to the polypeptide shown in SEQ ID NO: 11,
l) a polypeptide having at least 60%, at least 65%, at least 70%, at least 75% at least 80%, at least 85%, at least 90%, at least 95%, at least 98% or 100% sequence identity to the polypeptide shown in SEQ ID NO: 12,
m) a polypeptide having at least 60%, at least 65%, at least 70%, at least 75% at least 80%, at least 85%, at least 90%, at least 95%, at least 98% or 100% sequence identity to the polypeptide shown in SEQ ID NO: 13,
n) a polypeptide having at least 60%, at least 65%, at least 70%, at least 75% at least 80%, at least 85%, at least 90%, at least 95%, at least 98% or 100% sequence identity to the polypeptide shown in SEQ ID NO: 14,
o) a polypeptide having at least 60%, at least 65%, at least 70%, at least 75% at least 80%, at least 85%, at least 90%, at least 95%, at least 98% or 100% sequence identity to the polypeptide shown in SEQ ID NO: 15,
p) a polypeptide having at least 60%, at least 65%, at least 70%, at least 75% at least 80%, at least 85%, at least 90%, at least 95%, at least 98% or 100% sequence identity to the polypeptide shown in SEQ ID NO: 16,
q) a polypeptide having at least 60%, at least 65%, at least 70%, at least 75% at least 80%, at least 85%, at least 90%, at least 95%, at least 98% or 100% sequence identity to the polypeptide shown in SEQ ID NO: 17,
r) a polypeptide having at least 60%, at least 65%, at least 70%, at least 75% at least 80%, at least 85%, at least 90%, at least 95%, at least 98% or 100% sequence identity to the polypeptide shown in SEQ ID NO: 18,
s) a polypeptide having at least 60%, at least 65%, at least 70%, at least 75% at least 80%, at least 85%, at least 90%, at least 95%, at least 98% or 100% sequence identity to the polypeptide shown in SEQ ID NO: 19,
t) a polypeptide having at least 60%, at least 65%, at least 70%, at least 75% at least 80%, at least 85%, at least 90%, at least 95%, at least 98% or 100% sequence identity to the polypeptide shown in SEQ ID NO: 20,
u) a polypeptide having at least 60%, at least 65%, at least 70%, at least 75% at least 80%, at least 85%, at least 90%, at least 95%, at least 98% or 100% sequence identity to the polypeptide shown in SEQ ID NO: 21,
v) a polypeptide having at least 60%, at least 65%, at least 70%, at least 75% at least 80%, at least 85%, at least 90%, at least 95%, at least 98% or 100% sequence identity to the polypeptide shown in SEQ ID NO: 22, and
w) a polypeptide having at least 60%, at least 65%, at least 70%, at least 75% at least 80%, at least 85%, at least 90%, at least 95%, at least 98% or 100% sequence identity to the polypeptide shown in SEQ ID NO: 23,

wherein the protease is any one of those described above under the section “Polypeptides having protease activity”, and wherein the composition preferably comprises at least one cleaning component.

One embodiment relates to a cleaning composition, as defined herein, comprising a dispersin and a protease, wherein the dispersin comprises or consists of a polypeptide selected from the group of polypeptides comprising the amino acid sequence shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23 and wherein the protease comprises or consists of any one of the amino acid sequences described above under the section “Polypeptides having protease activity”.

One embodiment relates to a composition, as defined herein, comprising
a) at least 0.001 ppm of at least one polypeptide having dispersin activity, wherein the dispersin is selected from the group consisting of:
   i. a dispersin obtained from Terribacillus;
   ii. a dispersin obtained from Curtobacterium;
   iii. a dispersin obtained from Aggregatibacter;
   iv. a dispersin obtained from Haemophilus;
   v. a dispersin obtained from Actinobacillus;
   vi. a dispersin obtained from Lactobacillus;
   vii. a dispersin obtained from or Staphylococcus;
   viii. a polypeptide having hexosaminidase activity selected from: a polypeptide having at least 60%, at least 70%, at least 75%, at least 80%, at least 85%, at least 90%, at least 95%, at least 98%, at least 99% or 100% sequence identity to the polypeptide shown in SEQ ID NO: 1, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 2, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 3, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 4, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 5, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 6, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 7, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 8, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 9, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 10, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 11, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 12, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 13, a polypeptide having at least 60%, at least 70%, at least 75%, at least 80%, at least 85%, at least 90%, at least 95%, at least 98%, at least 99% or 100% sequence identityto the polypeptide shown in SEQ ID NO: 92, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 15, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 16, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 17, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 18, a polypeptide having at least 60%, at least 70%, at least 75%, at least 80%, at least 85%, at least 90%, at least 95%, at least 98%, at least 99% or 100% sequence identityto the polypeptide shown in SEQ ID NO: 19, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 20, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 21, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 22, a polypeptide having at least 60%, at least 70%, at least 75%, at least 80%, at least 85%, at least 90%, at least 95%, at least 98%, at least 99% or 100% sequence identity to the polypeptide shown in SEQ ID NO: 23, and
b) at least 0.01 ppm of at least one protease, wherein the protease is selected from any one of those described above under the section “Polypeptides having protease activity”, and, optionally,
c) at least one additional component e.g. a cleaning component, preferably selected from surfactants, builders, bleach components, polymers, dispersing agents and additional enzymes.

The protease and dispersin may be included in the cleaning composition of the present invention at a level of from 0.01 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, from 250 ppm to 500 ppm.

The dispersins above may be combined with proteases to form a blend to be added to the wash liquor solution. The concentration of the dispersin in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm.

The concentration of the protease in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm.

The dispersins may be combined with any of the proteases described above to form a blend to be added to a composition according to the invention.

One embodiment relates to a cleaning composition, as defined herein, comprising a dispersin and a protease, wherein the amount of dispersin in the composition is from 0.01 to 1000 ppm and the amount of protease is from 0.01 to 1000 ppm.

In addition to the protease and dispersin, the cleaning composition may further comprise at least one cleaning component. One embodiment relates to a cleaning composition, as defined herein, comprising a dispersin, a protease and at least one cleaning component, wherein the cleaning component is selected from surfactants, preferably anionic and/or nonionic, builders and bleach components.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 0.1% to about 15% or about 3% to about 10%.

“About”, as used herein in relation to a numerical value means said value ±10%, preferably ±5%. “About 5 wt %” thus means from 4.5 to 5.5 wt %, preferably from 4.75 to 5.25 wt %. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weight of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Preferred anionic surfactants are sulfate surfactants and in particular alkyl ether sulfates, especially C9-C15 alcohol ether sulfates, preferably ethoxylates or mixed ethoxylates/propoxylates, such as those with 1 to 30 EO, C12-C15 primary alcohol ethoxylate, such as those with 1 to 30 EO, C8-C16 ester sulfates and C10-C14 ester sulfates, such as mono dodecyl ester sulphates. Non-limiting examples of anionic surfactants include sulfates and sulfonates, in particular, linear alkylbenzenesulfonates (LAS), in particular C12-C13 alkyl benzene sulfonates, isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof. The anionic surfactants are preferably added to the detergent in the form of salts. Suitable cations in these salts are alkali metal ions, such as sodium, potassium and lithium and ammonium salts, for example (2-hydroxyethyl) ammonium, bis(2-hydroxyethyl) ammonium and tris(2-hydroxyethyl) ammonium salts. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof. Commercially available nonionic surfactants include Plurafac™, Lutensol™ and Pluronic™ range from BASF, Dehypon™ series from Cognis and Genapol™ series from Clariant.

In various embodiments, said surfactant preferably comprises at least one alkyl ether sulfate. Preferred alkyl ether sulfates are those of formula (I)

R¹—O-(AO)ₙ—SO₃⁻X⁺ (I).

In formula (I) R¹ represents a linear or branched, substituted or unsubstituted alkyl group, preferably a linear, unsubstituted alkyl group, more preferably a fatty alcohol moiety. Preferred R¹ moieties are selected from the group consisting of decyl, undecyl, dodecyl, tridecyl, tetradecyl, pentadecyl, hexadecyl, heptadecyl, octadecyl, nonadecyl, eicosyl moieties and mixtures thereof, wherein those groups with an even number of carbon atoms are preferred. Particularly preferred R¹ moieties are derived from C10-C18 fatty alcohols, such as those derived from coconut oil alcohols, tallow fatty alcohols, lauryl, myristyl, cetyl or stearyl alcohol or from C10-C20 oxoalcohols.

AO represents an ethyleneoxide (EO) or propyleneoxide (PO) group, preferably an ethyleneoxide group. The index n represents an integer from 1 to 50, preferably from 1 to 20 and more preferably from 1 to 10. Particularly preferably, n is 1, 2, 3, 4, 5, 6, 7 or 8. X represents a monovalent cation or the n-th part of an n-valent cation, preferred are alkali metal cations, specifically Na⁺ and K⁺, most preferably Na⁺. Further cations X⁺ may be selected from NH₄⁺, ½Zn²⁺, ½Mg²⁺, ½Ca²⁺, ½Mn²⁺, and combinations thereof.

In various preferred embodiments, the detergent compositions comprise an alkyl ether sulfate selected from fatty alcohol ether sulfates of formula (II)

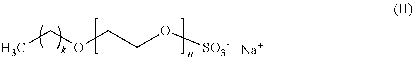

wherein k=9 to 19, and n=1, 2, 3, 4, 5, 6, 7 or 8. Preferred are C₁₀₋₁₆ fatty alcohol ether sulfates with 1-7 EO (k=9-15, n=1-7), such as the C₁₂₋₁₄ fatty alcohol ether sulfates with 1-3, particularly 2 EO (k=11-13, n=1-3 or 2), more particularly the sodium salts thereof. One specific embodiment thereof is lauryl ether sulfate sodium salt with 2 EO. The level of ethoxylation is an average value and can, for a specific compound, be an integer or fractional number.

In various embodiments, the surfactant comprises at least one alkyl benzene sulfonate. Said alkyl benzene sulfonate may be present alternatively to the above alkyl ether sulfate or, preferably, in addition to it.

Exemplary alkyl benzene sulfonates include, but are not limited to linear and branched alkyl benzene sulfonates, preferably linear alkyl benzene sulfonates. Exemplary compounds are those of formula (III)

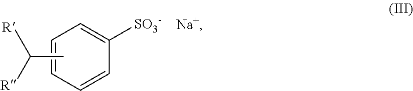

wherein R′ and R″ are independently H or alkyl and combined comprise 9 to 19, preferably 9 to 15 and more preferably 9 to 13 carbon atoms. Particularly preferred are dodecyl and tridecyl benzene sulfonates, in particular the sodium salts thereof.

In addition or alternatively, the compositions of the invention may further comprise one or more nonionic surfactants. Preferred nonionic surfactants are those of formula (IV)

R²—O-(AO)ₘ—H (IV),

wherein R² represents a linear or branched substituted or unsubstituted alkyl moiety, AO represents an ethylene oxide (EO) or propylene oxide (PO) group and m is an integer from 1 to 50.

In formula (IV) R² preferably represents a linear or branched, substituted or unsubstituted alkyl group, preferably a linear, unsubstituted alkyl group, particularly preferred a fatty alcohol group. Preferred groups of R² are selected from decyl, undecyl, dodecyl, tridecyl, tetradecyl, pentadecyl, hexadecyl, heptadecyl, octadecyl, nonadecyl, eicosyl groups and combinations thereof, wherein those groups with an even number of carbon atoms are preferred. Particularly preferred are R² groups derived from C₁₂-C₁₈ fatty alcohols, such as coconut oil alcohol, tallow oil alcohol, lauryl, myristyl, cetyl or stearyl alcohol or from C₁₀-C₂₀ oxoalcohols.

AO represents an ethyleneoxide (EO) or propyleneoxide (PO) group, preferably an ethyleneoxide group. The index m represents an integer from 1 to 50, preferably from 1 to 20 and more preferably from 1 to 6. Particularly preferably, m is 1, 2, 3, 4 or 5, most preferably 3-5, as higher degrees of ethoxylation may negatively influence viscosity and stability.

In various preferred embodiments, the detergent compositions comprise an alkyl ether selected from fatty alcohol ethers of formula (V)

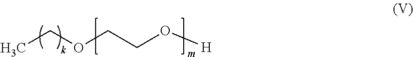

wherein k=11 to 19, m=1, 2, 3, 4, 5, 6, 7 or 8. Preferred are C₁₂₋₁₈ fatty alcohols with 1-6 EO (k=11-17, m=1-5 in formula (V)). More preferred are C₁₂₋₁₄ alcohols having 1-5 EO, most preferred are C₁₂₋₁₄ alkyl ethers with 3-5 EO, in particular lauryl ether with 5 EO.

The detergent compositions may further include other nonionic surfactants, such as alkyl glucosides of the general formula RO(G)ₓ, where R is a primary linear or 2-methyl-branched aliphatic radical containing 8 to 22 and preferably 12 to 18 carbon atoms and G stands for a glucose unit. The degree of oligomerization x, which indicates the distribution of monoglucosides and oligoglucosides, is a number of 1 to 10 and preferably a number of 1.2 to 1.4.

In various embodiments, the composition comprises at least two anionic surfactants, e.g. at least one alkyl ether sulfate and preferably at least one alkyl benzene sulfonate, and optionally an alkyl ether.

Suitable amphoteric surfactants comprise betains. Preferred betaines are the alkylbetaines, the alkylamidobetaines, the imidazolinium betaines, the sulfobetaines (INCI Sultaines) and the phosphobetaines. Examples of suitable betaines and sulfobetaines are the following compounds designated as INCI: almondamidopropyl betaines, apricotam idopropyl betaines, avocadamidopropyl betaines, babassuamidopropyl betaines, behenamide idopropyl betaines, behenyl betaines, betaines, canola idopropyl betaines, caprylic/capram idopropyl betaines, carnitines, cetyl betaines, cocamidoethyl betaines, cocamidopropyl betaines, cocam idopropyl hydroxysultaines, cocobetaines, coco-hydroxysultaines, coco/oleam idopropyl betaines, coco-sultaines, decyl betaines, dihydroxyethyl oleyl glycinates, dihydroxyethyl soy glycinates, dihydroxyethyl stearyl glycinates, dihydroxyethyl tallow glycinates, dimethicones propyl PG Betaines, erucam idopropyl hydroxysultaines, hydrogenated tallow betaines, isostearam idopropyl betaines, lauram idopropyl betaines, lauryl betaines, lauryl hydroxysultaine, lauryl sultaines, milkamidopropyl betaines, minkam idopropyl betaines, myristamine idopropyl betaines, myristyl betaines, oleam idopropyl betaines, oleam idropy Hydroxysultain, Oleyl Betaine, Olivamidopropyl Betaine, Palmam Idopropyl Betaine, Palm Itam Idopropyl Betaine, Palmitoyl Carnitine, Palm Kernelamidopropyl Betaine, Polytetrafluoroethylene Acetoxypropyl Betaine, Ricinoleam Idopropyl Betaine, Sesamidopropyl Betaine, Soyamidopropyl Betaine, Stearam Idopropyl Betaine, Stearyl Betaine, Tallowam Idopropyl Betaine, Tallowamidopropyl Hydroxysultaine, Tallow Betaine, Tallow Dihydroxyethyl Betaine, Undecylenamidopropyl Betaine and Wheat Germamidopropyl Betaine. A preferred betaine is, for example, cocamidopropyl betaine (cocoamidopropylbetaine). The betaines are particularly preferred for dishwashing compositions, most preferably hand dishwashing detergent compositions.

Further suitable surfactants include the amine oxides. The amine oxides suitable in accordance with the invention include alkylamine oxides, in particular alkyldimethylamine oxides, alkylamidoamine oxides and alkoxyalkylamine oxides. Examples of suitable amine oxides are the following compounds designated as INCI: Almond amidopropylamine oxides, Babassu amidopropylamine oxides, Behenamine oxides, Cocamidopropyl Amine oxides, Cocamidopropylamine oxides, Cocamine oxides, Coco-Morpholine oxides, Decylamine oxides, Decyltetradecylamine oxides, Diaminopyrimidine oxides, Dihydroxyethyl C8-10 alkoxypropylamines oxides, Dihydroxyethyl C9-11 alkoxypropylamines oxides, dihydroxyethyl C12-15 alkoxypropylamines oxides, dihydroxyethyl cocamine oxides, dihydroxyethyl lauramine oxides, dihydroxyethyl stearamines oxides, dihydroxyethyl tallowamine oxides, hydrogenated palm kernel amine oxides, hydrogenated tallowamine oxides, hydroxyethyl hydroxypropyl C12-15 alkoxypropylamines oxides, isostearamidopropylamines Oxides, isostearamidopropyl morpholine oxides, lauram idopropylamine oxides, lauramine oxides, methyl morpholine oxides, milkamidopropyl amine oxides, mincamidopropylamine oxides, myristamine idopropylamine oxides, myristamine oxides, myristyl/cetyl amines Oxides, Oleam idopropylamine oxides, Oleamine oxides, OI ivam idopropylam ine oxides, Palmitamidopropylamine oxides, Palmitamine oxides, PEG-3 Lauramine oxides, Potassium dihydroxyethyl Cocamine oxides phosphates, Potassium Trisphosphonomethylamine oxides, Sesamidopropylamine oxides, Soyamidopropylamine oxides, Stearam idopropylam ine oxides, stearamines Oxides, Tallowam idopropylam ine oxides, Tallowamine oxides, Undecylenamidopropylamine oxides and Wheat Germam idopropylam ine oxides. A preferred amine oxide is, for example, cocamidopropylamine oxides (cocoamidopropylamine oxide).

For automatic dishwashing applications, low-foaming nonionic surfactants are preferably used, in particular alkoxylated, especially ethoxylated, low-foaming nonionic surfactants. With particular preference, the automatic dishwashing detergents contain nonionic surfactants from the group of the alkoxylated alcohols. Particular preference is given to nonionic surfactants which have a melting point above room temperature. Nonionic surfactants having a melting point above 20° C., preferably above 25° C., more preferably between 25 and 60° C. and especially between 26.6 and 43.3° C., are particularly preferred. Preferably used surfactants are those from the groups of alkoxylated nonionic surfactants, in particular the ethoxylated primary alcohols and mixtures of these surfactants with structurally more complex surfactants such as polyoxypropylene/polyoxyethylene/polyoxypropylene ((PO/EO/PO) surfactants). Such (PO/EO/PO) nonionic surfactants are also characterized by good foam control. Particularly preferred nonionic surfactants are those containing alternating ethylene oxide and different alkylene oxide units. Among these, in turn, surfactants with EO-AO-EO-AO blocks are preferred, with one to ten EO or AO groups before one block from the other group follows. Exemplary nonionic surfactants are those having a C9-alkyl group with 1 to 4 ethylene oxide units followed by 1 to 4 propylene oxide units, followed by 1 to 4 ethylene oxide units followed by 1 to 4 propylene oxide units. Preference is given in particular to end-capped, poly (oxyalkylated) nonionic surfactants with the end-cap being a linear or branched, saturated or unsaturated, aliphatic or aromatic hydrocarbon radical R having 1 to 30 carbon atoms. The alkyl groups may also comprise hydroxyl groups. The group of these nonionic surfactants include, for example, the C4-22 fatty alcohol (EO)₁₀₋₅₀-2-hydroxyalkyl ethers, in particular also the C8-12 fatty alcohol (EO)₂₂-2-hydroxydecyl ethers and the C4-22 fatty alcohol (EO)₄₀₋₈₀-2-hydroxyalkyl ethers.

Builders and Co-Builders

The cleaning composition may contain about 0-65% by weight, such as about 5% to about 50%, such as about 0.5% to about 20% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

Generally and if not indicated otherwise, the builder may be preferably selected from citrate, carbonate, silicate, aluminosilicate (zeolite) and combinations thereof. Suitable builders also include phosphonates, polyphosphonates, bicarbonates, borates, and further polycarboxylates. Citrate builders, e.g., citric acid and soluble salts thereof (particularly sodium salt), are particularly suitable water-soluble organic builders. Citrates can be used in combination with zeolite, silicates like the BRITESIL types, and/or layered silicate builders. The builder and/or co-builder may be any chelating agent that forms water-soluble complexes with Ca and Mg. Any builder and/or co-builder known in the art for use in cleaning detergents may be utilized. Non-limiting examples of builders include zeolites, in particular zeolite A or P or X, carbonates such as sodium carbonate, soluble silicates such as sodium metasilicate, layered silicates (e.g., SKS-6 from Hoechst), and (carboxymethyl)inulin (CMI), and combinations thereof. Further non-limiting examples of builders include aminocarboxylates, aminopolycarboxylates, and alkyl- or alkenylsuccinic acid. Additional specific examples include 2,2′,2-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycine-N,N-diacetic acid (MGDA), glutamic acid-N,N-diacetic acid (GLDA), 1-hydroxyethane-1,1-diphosphonic acid, N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(sulfomethyl)aspartic acid (SMAS), N-(2-sulfoethyl)-aspartic acid (SEAS), N-(sulfomethylglutamic acid (SMGL), N-(2-sulfoethyl)-glutamic acid (SEGL), N-methyliminodiacetic acid (MIDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and N′-(2-hydroxyethyl)ethylenediamine-N,N,N′-triacetic acid (HEDTA), diethanolglycine (DEG), and combinations and salts thereof. Phosphonates suitable for use herein include 1-hydroxyethane-1,1-diphosphonic acid (HEDP), ethylenediaminetetrakis (methylenephosphonic acid) (EDTMPA), diethylenetriaminepentakis (methylenephosphonic acid) (DTMPA or DTPMPA or DTPMP), nitrilotris (methylenephosphonic acid) (ATMP or NTMP), 2-phosphonobutane-1,2,4-tricarboxylic acid (PBTC), hexamethylenediaminetetrakis (methylenephosphonic acid) (HDTMP). Particularly preferred are HEDP and DTPMP.

Suitable silicates are crystalline, layered sodium silicates of the general formula NaMSiₓO₂₊₁*yH₂O, wherein M is sodium or H, x a number of from 1.9 to 4 and y a number of from 0 to 20 and x is preferably 2, 3 or 4. Such silicates are for example disclosed in EP 0 164 514. Preferred are silicates in which M is sodium and is 2 or 3. Particularly preferred are β- and δ-sodium disilicate Na₂Si₂O₅*yH₂O.

Although not preferred, the compositions may also comprise phosphates, diphosphates (pyrophosphates) and/or triphosphates such as sodium triphosphate (STP or STPP). It is however preferred that all compositions disclosed herein are phosphate-free, i.e. do not contain deliberately added phosphate, in particular the phosphate content is below 1 wt %, more preferably less than 0.5 wt %, even more preferably less than 0.1 wt %, relative to the total weight of the composition. In alternative embodiments, the invention also relates to phosphate-free cleaning compositions in general that contain the polypeptides of the invention. In one embodiment, the invention thus features a phosphate-free cleaning composition comprising any one or more of the polypeptides having hexosaminidase activity disclosed herein.

If not indicated otherwise, the composition may also contain 0-50% by weight, such as about 5% to about 30%, of a detergent co-builder. The composition may include a co-builder alone, or in combination with a builder, for example a zeolite builder. Non-limiting examples of co-builders include homopolymers of polyacrylates or copolymers thereof, such as poly (acrylic acid) (PAA) or copoly (acrylic acid/maleic acid) (PAA/PMA) or polyaspartic acid. Further exemplary builders and/or co-builders are described in, e.g., WO 09/102854, U.S. Pat. No. 5,977,053.

Preferred as co-builders are acrylate-containing water-soluble polymers, such as alkali metal salts of polyacrylic acid or polymethacrylic acid, for example those having a molecular weight M_{w} in the range of 600 to 750,000 g/mol, as determined by gel permeation chromatography (GPC) according to DIN 55672-1:2007-08 with THE as an eluent.

Preferred polymers are polyacrylates with a molecular weight M_{w} of 1,000 to 15,000 g/mol, more preferred, due to their solubility, are short-chain polyacrylates with a molecular weight M_{w} of 1,000 to 10,000 g/mol, most preferred from 1,000 to 5,000 g/mol.

Preferred acrylates for use in the present invention are alkali metal salts of polymers of acrylic acid, preferably the sodium salts, in particular those with molecular weights in the range of 1,000 to 10,000 g/mol or 1,000 to 5,000 g/mol. Suitable acrylates are commercially available, for example under the tradename Acusol® from Dow Chemical. Suitable are also copolymers of acrylates, in particular those of acrylic acid and methacrylic acid, and acrylic acid or methacrylic acid and maleic acid.

In preferred embodiments, the compositions of the invention comprise a sulfopolymer, preferably a copolymer comprising an ethylenically unsaturated sulfonate/sulfonic acid as a co-monomer. Particularly suitable are monomers of allyl sulfonic acids, such as allyloxybenzene sulfonic acid and methallyl sulfonic acid. Particularly preferred sulfonic acid group-containing monomers are 1-acrylamido propane sulfonic acid-1,2-acrylamido-2-propanesulfonic acid, 2-acrylamido-2-methyl-1-propanesulfonic acid, 2-methacrylamido-2-methyl-1-propanesulfonic acid, 3-methacrylamido-2-hydroxy-propanesulfonic acid, allylsulfonic acid, methallylsulfonic acid, allyloxybenzenesulfonic acid, methallyloxybenzolsulfonsaure, 2-hydroxy-3-(2-propenyloxy) propanesulfonic acid, 2-methyl-2-propenl-sulfonic acid, styrenesulfonic acid, vinylsulfonic acid, 3-sulfopropyl, sulfomethacrylamide, sulfomethylmethacrylamide and mixtures of said acids or their water-soluble salts.

The sulfopolymers are preferably copolymers of the afore-described monomers with unsaturated carboxylic acids, Especially preferred unsaturated carboxylic acids are acrylic acid, methacrylic acid, ethacrylic acid, chloroacrylic acid, alpha-cyanoacrylic acid, crotonic acid, alpha-phenyl-acrylic acid, maleic acid, maleic anhydride, fumaric acid, itaconic acid, citraconic acid, methylenemalonic acid, sorbic acid, cinnamic acid or mixtures thereof. Usable are of course also the unsaturated dicarboxylic acids.

Preferred are copolymers with acrylates, in particular with acrylic acid and methacrylic acid, and acrylic acid or methacrylic acid and maleic acid.

Such polymers are, for example, commercially available under the trade names Acusol® 590 or Acusol®588 from Dow Chemical.

In one embodiment of the invention, the cleaning compositions of the invention comprise a polypeptide as defined herein and at least one sulfopolymer, as defined above. Such compositions are preferably dishwashing compositions.

In one preferred embodiment, the builder is a non-phosphorus based builder such as citric acid and/or methylglycine-N,N-diacetic acid (MGDA) and/or glutamic-N,N-diacetic acid (GLDA) and/or salts thereof.

Bleaching Systems

The cleaning composition, as defined herein, may contain 0-30% by weight, such as about 1% to about 20%, such as about 0.01% to about 10% of a bleaching system. Any bleaching system comprising components known in the art for use in cleaning detergents may be utilized. Suitable bleaching system components include sources of hydrogen peroxide; sources of peracids; and bleach catalysts or boosters.

Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

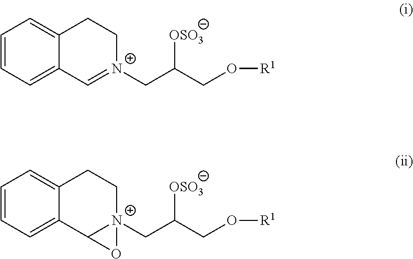

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Metal Care Agents

Metal care agents may prevent or reduce the tarnishing, corrosion or oxidation of metals, including aluminium, stainless steel and non-ferrous metals, such as silver and copper. Suitable examples include one or more of the following:

(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups (e.g., C1-C20- alkyl groups) and hydroxyl, thio, phenyl or halogen such as fluorine, chlorine, bromine and iodine.

(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one embodiment, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate.;

(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

Hydrotropes

The cleaning composition, as defined herein, may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Polymers

The cleaning composition, as defined herein, may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

Fabric Hueing Agents

The cleaning compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

Enzymes

The cleaning composition, as defined herein, may comprise one or more additional enzymes such as one or more enzyme selected from lipases, cutinases, amylases, carbohydrases, cellulases, pectinases, mannanases, arabinases, galactanases, xylanases, oxidases, e.g., a laccase and/or peroxidases. In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259. Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544. Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147). Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades). Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases

Suitable amylases include alpha-amylases and/or a glucoamylases and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444. Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:
M197T;
H156Y+A181T+N190F+A209V+Q264S; or
G48A+T491+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184. Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264. Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201 L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T1311+T1651+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:
E187P+1203Y+G476K
E187P+1203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128I K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions: N21 D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme PIus™, Natalase™, Liquozyme X and BAN™ (from Novozymes A/S), and Rapidase™ Purastar™/Effectenz™, Powerase, Preferenz Si000, Preferenz S100 and Preferenz S110 (from Genencor International Inc./DuPont).

Peroxidases/Oxidases

Typically, a peroxidase as defined herein is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257. Commercially available peroxidases include Guardzyme™ (Novozymes A/S).

A suitable peroxidase includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. Preferably, the haloperoxidase is a vanadium haloperoxidase, i.e., a vanadate-containing haloperoxidase. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

A suitable oxidase includes in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular, a laccase derived from Coprinopsis cinerea, as disclosed in WO 97/08325; or from Myceliophthora thermophila, as disclosed in WO 95/33836.

Dispersants

The cleaning compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are, for example, described in Powdered Detergents, Surfactant science series volume 71, Marcel Dekker, Inc.

Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Aquent

The cleaning compositions of the present invention will preferably also contain additional components that may tint articles being cleaned, such as fluorescent whitening agent or optical brighteners. Where present, the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

Soil Release Polymers

The cleaning compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents

The cleaning compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Rheology Modifiers

The cleaning compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

Formulation of Detergent Products

The cleaning composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. In a specific embodiment, the present invention provides a detergent additive comprising one or more enzymes as described herein. The cleaning composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1. Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

Granular Cleaning Formulations

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The dispersin may be formulated as a granule, for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulate for the detergent industry is disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt % zeolite (anhydrous basis); and (c) less than 10 wt % phosphate salt (anhydrous basis), wherein said enzyme co-granule comprises from 10 to 98 wt % moisture sink component and the composition additionally comprises from 20 to 80 wt % detergent moisture sink component. The multi-enzyme co-granule may comprise an enzyme of the invention and one or more enzymes selected from the group consisting of proteases, lipases, cellulases, xyloglucanases, perhydrolases, peroxidases, lipoxygenases, laccases, hemicellulases, proteases, cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, mannanases and amylases, and mixtures thereof. WO 2013/188331 also relates to a method of treating and/or cleaning a surface, preferably a fabric surface comprising the steps of (i) contacting said surface with the detergent composition as claimed and described herein in aqueous wash liquor, (ii) rinsing and/or drying the surface.

An embodiment of the invention relates to an enzyme granule/particle comprising the dispersin and protease. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm. The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606. The coating may be applied in an amount of at least 0.1% by weight of the core, e.g., at least 0.5%, 1% or 5%. The amount may be at most 100%, 70%, 50%, 40% or 30%. The coating is preferably at least 0.1 μm thick, particularly at least 0.5 μm, at least 1 μm or at least 5 μm. In a one embodiment, the thickness of the coating is below 100 μm. In another embodiment, the thickness of the coating is below 60 μm. In an even more particular embodiment, the total thickness of the coating is below 40 μm. The coating should encapsulate the core unit by forming a substantially continuous layer. A substantially continuous layer is to be understood as a coating having few or no holes, so that the core unit it is encapsulating/enclosing has few or none uncoated areas. The layer or coating should be homogeneous in thickness. The coating can further contain other materials as known in the art, e.g., fillers, antisticking agents, pigments, dyes, plasticizers and/or binders, such as titanium dioxide, kaolin, calcium carbonate or talc. A salt coating may comprise at least 60% by weight w/w of a salt, e.g., at least 65%, at least 70%, at least 75%, at least 80%, at least 85%, at least 90%, at least 95% or at least 99% by weight w/w. The salt may be added from a salt solution where the salt is completely dissolved or from a salt suspension wherein the fine particles is less than 50 μm, such as less than 10 μm or less than 5 μm. The salt coating may comprise a single salt or a mixture of two or more salts. The salt may be water soluble, and may have a solubility at least 0.1 grams in 100 g of water at 20° C., preferably at least 0.5 g per 100 g water, e.g., at least 1 g per 100 g water, e.g., at least 5 g per 100 g water. The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{200° C.}=81.1%), KCl (CH_{200° C.}=85%), K₂HPO₄ (CH_{200° C.}=92%), KH₂PO₄ (CH_{20° C.}=96.5%), KNO₃ (CH_{200° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate. The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄.7H₂O), zinc sulfate heptahydrate (ZnSO₄.7H₂O), sodium phosphate dibasic heptahydrate (Na₂HPO₄.7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

One embodiment of the present invention provides a granule, which comprises:
(a) a core comprising a dispersin and a protease according to the invention, and
(b) optionally a coating consisting of one or more layer(s) surrounding the core.

One embodiment of the invention relates to a granule, which comprises:
(a) a core comprising a dispersin and a protease, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23, and
(b) optionally a coating consisting of one or more layer(s) surrounding the core.

Uses

The present invention is also directed to methods for using the compositions of the invention in e.g. laundry/textile/fabric (House hold laundry washing, Industrial laundry washing) and hard surface cleaning (ADW, car wash, Industrial surface). The cleaning e.g. detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. In a specific embodiment, the present invention provides a detergent additive comprising one or more enzymes as described herein.

The compositions of the invention comprise a blend of dispersin and protease and effectively reduce or remove organic components, such as protein and PNAG from surfaces such as textiles and hard surfaces e.g. dishes.

One embodiment of the invention relates to the use of a composition comprising a dispersin and protease for reduction of redeposition. One embodiment of the invention relates to the use of a cleaning composition, as defined herein, comprising a dispersin and protease for reduction of redeposition.

One embodiment of the invention relates to the use of a cleaning composition, as defined herein, comprising a dispersin and protease for reduction of redeposition when the cleaning composition is applied in e.g. laundry process. One embodiment of the invention relates to the use of a cleaning composition, as defined herein, comprising a dispersin and protease for reduction of redeposition on an item e.g. textile. In one embodiment, the composition is an anti-redeposition composition.

One embodiment of the invention relates to the use of a cleaning composition, as defined herein, comprising a dispersin and protease for reduction of redeposition, wherein the protease is any one of those described above under the section “Polypeptides having protease activity”.

One embodiment of the invention relates to the use of a cleaning composition, as defined herein, comprising a dispersin and protease for reduction of redeposition, wherein the protease is any one of those described above under the section “Polypeptides having protease activity” and wherein the dispersin is obtained from Terribacillus or Curtobacterium or Aggregatibacter or Haemophilus or Actinobacillus or Lactobacillus or Staphylococcus.

One embodiment of the invention relates to the use of a cleaning composition, as defined herein, comprising a dispersin and protease for reduction of redeposition, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23.

The compositions of the invention may comprise a blend of dispersin and protease and effectively reduce or limit malodor of e.g. textiles or hard surfaces such as dishes.

One embodiment of the invention relates to the use of a composition comprising a dispersin and protease for reduction of malodor. One embodiment of the invention relates to the use of a cleaning composition, as defined herein, comprising a dispersin and protease for reduction of malodor.

One embodiment of the invention relates to the use of a cleaning composition, as defined herein, comprising a dispersin and protease for reduction of malodor when the cleaning composition is applied in e.g. laundry process. One embodiment of the invention relates to the use of a cleaning composition, as defined herein, comprising a dispersin and protease for reduction of malodor on an item e.g. textile.

One embodiment of the invention relates to the use of a cleaning composition, as defined herein, comprising a dispersin and protease for reduction of malodor, wherein the protease is any one of those described above under the section “Polypeptides having protease activity”.

One embodiment of the invention relates to the use of a cleaning composition, as defined herein, comprising a dispersin and protease for reduction of malodor, wherein the protease is any one of those described above under the section “Polypeptides having protease activity” and wherein the dispersin is obtained from Terribacillus or Curtobacterium or Aggregatibacter or Haemophilus or Actinobacillus or Lactobacillus or Staphylococcus.

One embodiment of the invention relates to the use of a cleaning composition, as defined herein, comprising a dispersin and protease for reduction of malodor, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23.

The compositions of the invention comprise a blend of dispersin and protease and improve whiteness of textile. One embodiment of the invention relates to the use of a composition, as defined herein, comprising a dispersin and protease for improvement of whiteness of an item e.g. a textile. One embodiment of the invention relates to the use of a cleaning composition, as defined herein, comprising a dispersin and protease for improvement of whiteness. One embodiment of the invention relates to the use of a cleaning composition, as defined herein, comprising a dispersin and protease improve whiteness when the cleaning composition is applied in e.g. laundry process. One embodiment of the invention relates to the use of a cleaning composition, as defined herein, comprising a dispersin and protease improve whiteness on an item e.g. textile.

One embodiment of the invention relates to the use of a cleaning composition, as defined herein, comprising a dispersin and protease for improvement of whiteness, wherein the protease is any one of those described above under the section “Polypeptides having protease activity”.

One embodiment of the invention relates to the use of a cleaning composition, as defined herein, comprising a dispersin and protease for improvement of whiteness, wherein the protease is any one of those described above under the section “Polypeptides having protease activity” and wherein the dispersin is obtained from Terribacillus or Curtobacterium or Aggregatibacter or Haemophilus or Actinobacillus or Lactobacillus or Staphylococcus.

One embodiment of the invention relates to the use of a cleaning composition, as defined herein, comprising a dispersin and protease for improvement of whiteness, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO:1 or 2 or 3 or 4 or 5 or 6 or 7 or 8 or 9 or 10 or 11 or 12 or 13 or 14 or 15 or 16 or 17 or 18 or 19 or 20 or 21 or 22 or 23.

The compositions of the invention comprise a blend of dispersin and protease and effectively reduce or remove organic components, such as protein and PNAG from surfaces such as textiles and hard surfaces e.g. dishes. One embodiment of the invention relates to the use of a cleaning composition, as defined herein, comprising a dispersin and a protease for reduction or removal of biofilm and components of biofilm, such as PNAG and protease, of an item, wherein the item is a textile or a hard surface.

One embodiment of the invention relates to the use of a cleaning composition, as defined herein, comprising a dispersin, a protease and at least one cleaning component for deep cleaning of an item, wherein the item is a textile or a surface.

One embodiment of the invention relates to the use of a composition, as defined herein, comprising a dispersin and protease for reduction or removal of biofilm compounds such as PNAG and protease of an item. One embodiment of the invention relates to the use of a cleaning composition, as defined herein, comprising a dispersin and protease for reduction or removal of biofilm compounds such as PNAG and protease of an item such as textile. One embodiment of the invention relates to the use of a cleaning composition, as defined herein, comprising a dispersin and protease for deep cleaning e.g. reduction or removal of biofilm compounds such as PNAG and protease when the cleaning composition is applied in e.g. laundry process.

One embodiment of the invention relates to the use of a cleaning composition, as defined herein, comprising a dispersin and protease for deep cleaning of an item, wherein the protease is any one of those described above under the section “Polypeptides having protease activity”.

One embodiment of the invention relates to the use of a cleaning composition, as defined herein, comprising a dispersin and protease for deep cleaning of an item, wherein the protease is any one of those described above under the section “Polypeptides having protease activity” and wherein the dispersin is obtained from Terribacillus or Curtobacterium or Aggregatibacter or Haemophilus or Actinobacillus or Lactobacillus or Staphylococcus.

One embodiment of the invention relates to the use of a cleaning composition, as defined herein, comprising a dispersin and protease for deep cleaning of an item, wherein the protease is any one of those described above under the section “Polypeptides having protease activity” and wherein the dispersin has at least 60%, at least 65%, at least 70%, at least 75%, at least 80%, at least 85%, at least 90%, at least 95%, at least 98% or 100% sequence identity to the amino acid sequence shown in SEQ ID NO: 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23.

The invention further relates to a method of deep cleaning of an item, wherein the item may be textile or hard surface preferably a textile.

The invention further relates to a method of deep cleaning of an item, comprising the steps of:
a) contacting the item with a cleaning composition according to the invention; and
b) optionally rinsing the item, wherein the item is preferably a textile.

One embodiment of the invention relates to a method of deep cleaning on an item, comprising the steps of:
a) contacting the item with a composition of the invention comprising an enzyme mixture comprising a dispersin and a protease as defined herein; and
b) optionally rinsing the item, wherein the item is preferably a textile.

### Definitions

“Biofilm” is produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community.

On laundry and textiles, examples of biofilm-producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. On hard surfaces, examples of biofilm-producing bacteria can be found among the following species: Acinetobacter sp., Aeromicrobium sp., Brevundimonas sp., Microbacterium sp., Micrococcus luteus, Pseudomonas sp., Staphylococcus epidermidis, Staphylococcus aureus and Stenotrophomonas sp. In one embodiment, the biofilm producing strain is Brevundimonas sp. In one embodiment, the biofilm producing strain is Pseudomonas alcaliphila or Pseudomonas fluorescens. In one embodiment, the biofilm producing strain is Staphylococcus aureus.

By the term “deep cleaning” is meant reduction, disruption or removal of components which may be comprised in organic matter, e.g. biofilm, such as polysaccharides, proteins, DNA, soil or other components present in the organic matter.

Cleaning composition: The term “cleaning composition” refers to compositions that find use in the removal of undesired compounds from items to be cleaned, such as textiles. The cleaning composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzymes, the cleaning composition may contain one or more additional enzymes (such as amylases, lipases, cutinases, cellulases, endoglucanases, xyloglucanases, pectinases, pectin lyases, xanthanases, peroxidases, haloperoxygenases, catalases and mannanases, or any mixture thereof), and/or cleaning components e.g. detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers, all of which are as defined herein.

The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides. Textile care benefits, which are not directly related to catalytic stain removal or prevention of redeposition of soils, are also important for enzyme detergency benefits. Examples of such textile care benefits are prevention or reduction of dye transfer from one textile to another textile or another part of the same textile (an effect that is also termed dye transfer inhibition or anti-backstaining), removal of protruding or broken fibers from a textile surface to decrease pilling tendencies or remove already existing pills or fuzz (an effect that also is termed anti-pilling), improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.”

The term “hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

The term “wash performance” is used as an enzyme's ability to remove stains present on the object to be cleaned during e.g. wash or hard surface cleaning.

The term “whiteness” is defined herein as a measure of the greying or yellowing of a textile. Loss of whiteness may be due to removal of optical brighteners/hueing agents. Greying and yellowing can be due to soil redeposition, body soils, colouring from e.g. iron and copper ions or dye transfer. Loss of whiteness can be related to one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

By the term “malodor” is meant an odor which is not desired on clean items. The cleaned item should smell fresh and clean without malodors adhered to the item. One example of malodor is due to compounds with an unpleasant smell, which may be produced by microorganisms. Another example of unpleasant smells can be sweat or body odor adhered to an item which has been in contact with human or animal. Another example of malodor can be the odor from spices, which sticks to items for example curry or other exotic spices which smells strongly.

The term “mature polypeptide” means a polypeptide in its final form following translation and any post-translational modifications, such as N-terminal processing, C-terminal truncation, glycosylation, phosphorylation, etc.

The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well.

The term “variant” means a polypeptide having the activity of the parent or precursor polypeptide and comprising an alteration, i.e., a substitution, insertion, and/or deletion, at one or more positions compared to the precursor or parent polypeptide. A substitution means replacement of the amino acid occupying a position with a different amino acid; a deletion means removal of the amino acid occupying a position; and an insertion means adding an amino acid adjacent to and immediately following the amino acid occupying a position.

Sequence identity: The relatedness between two amino acid sequences or between two nucleotide sequences is described by the parameter “sequence identity”. For purposes of the present invention, the sequence identity between two amino acid sequences can be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows: (Identical Residues×100)/(Length of Alignment−Total Number of Gaps in Alignment) Nomenclature: For purposes of the present invention, the nomenclature [E/Q] means that the amino acid at this position may be a glutamic acid (Glu, E) or a glutamine (Gln, Q). Likewise, the nomenclature [V/G/A/I] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

For an amino acid substitution, the following nomenclature is used: Original amino acid, position, substituted amino acid. For example, the substitution of a threonine at position 220 with alanine is designated as “T220A”. Multiple substitutions may be separated by addition marks (“+”), e.g., “T220A+G229V”, representing substitutions at positions 220 and 229 of threonine (T) with alanine (A) and glycine (G) with valine (V), respectively. Multiple substitutions may alternatively be listed with individual mutations separated by a space or a comma. Alternative substitutions in a particular position may be indicated with a slash (“/”). For example, substitution of threonine in position 220 with either alanine, valine or leucine many be designated “T220AV/L”.

1. A cleaning composition comprising a dispersin, a protease, and, optionally, at least one cleaning component, wherein the protease is selected from
(1) a protease comprising an amino acid sequence having at least 90% sequence identity to the amino acid sequence set forth in SEQ ID NO:24 over its entire length and comprising the amino acid substitution R99E in combination with at least two further amino acid substitutions selected from the group consisting of S3T, V4I and V199I, wherein positional numbering is according to SEQ ID NO:24; or
(2) a protease comprising an amino acid sequence having at least 90% sequence identity to SEQ ID NO:27, wherein the protease variant has a glutamic acid residue (E) in position 101, and wherein the protease variant further comprises one or more substitutions selected from S156D; L262E; Q137H; S3T; R45E,D; P55N; T58W,Y,L; Q59D,M,N,T; G61D,R; S87E; G97S; A98D,E,R; S106A,W; N117E; H120V,D,K,N; S124M; P129D; E136Q; S143W; S161T; S163A,G; Y171L; A172S; N185Q; V199M; Y209W; M222Q; N238H; V244T; N261T; and L262N,Q,D; or
(3) a protease comprising an amino acid sequence having at least 90%, sequence identity to the amino acid sequence set forth in SEQ ID NO:30 over its entire length and comprising an amino acid substitution in at least one position corresponding to positions 12, 43, 122, 127, 154, 156, 160, 211, 212 and 222 of SEQ ID NO:30: or
(4) a protease comprising an amino acid sequence having at least 90%, sequence identity to the amino acid sequence set forth in SEQ ID NO:39 over its entire length and comprising (i) at least two of the amino acid substitutions 3T, 4I, 99E and 199I at the positions corresponding to positions 3, 4, 99 and 199 of SEQ ID NO:39 and (ii) in at least one position corresponding to positions 74, 136, 143, 154, 161, 163, 171, 200, 203, 209, 212 or 256 of SEQ ID NO:39.

2. The cleaning composition of claim 1, wherein the protease is selected from:
(1) a protease comprising an amino acid sequence having at least 90% sequence identity to SEQ ID NO:27, wherein the protease variant has a glutamic acid residue (E) in position 101, and wherein the protease variant further comprises one or more substitutions selected from R45E,D,Q; Q59D; T58L; G61D; S87E; G97S; A98E; S106A; N117E; H120D,K,V; P129D; E136Q; Q137H; S156D; S161T; S163A,G; V199M; M222Q; N261T; and L262E,Q,N;
(2) a protease comprising the amino acid sequence of SEQ ID NO:27 and having one of the following amino acid substitutions or set of amino acid substitutions:
SEQ ID NO:27+S3T;
SEQ ID NO:27+R45E/D;
SEQ ID NO:27+P55N;
SEQ ID NO:27+T58W/Y/L;
SEQ ID NO:27+Q59D/M/N/T;
SEQ ID NO:27+G61D/R;
SEQ ID NO:27+S87E;
SEQ ID NO:27+G97S;
SEQ ID NO:27+A98D/E/R;
SEQ ID NO:27+S106A/W;
SEQ ID NO:27+N117E;
SEQ ID NO:27+H120V/D/K/N;
SEQ ID NO:27+S124M;
SEQ ID NO:27+P129D;
SEQ ID NO:27+E136Q;
SEQ ID NO:3+S143W;
SEQ ID NO:3+S161T;
SEQ ID NO:3+S163A/G;
SEQ ID NO:3+Y171L;
SEQ ID NO:27+A172S;
SEQ ID NO:27+N185Q;
SEQ ID NO:27+V199M;
SEQ ID NO:27+Y209W;
SEQ ID NO:27+M222Q;
SEQ ID NO:27+N238H;
SEQ ID NO:27+V244T;
SEQ ID NO:27+N261 T;
SEQ ID NO:27+L262N/Q/D/E;
SEQ ID NO:27+N76D+S163G+N238E;
SEQ ID NO:27+S156D+L262E;
SEQ ID NO:27+N238E+L262E;
SEQ ID NO:27+S3T+N76D+S156D+Y209W;
SEQ ID NO:27+H120D+S163G+N261D;
SEQ ID NO:27+S163G+N128Q+N238E+L262E;
SEQ ID NO:27+K27Q+H120D+S163G+N261D;
SEQ ID NO:27+V104T+H120D+S156D+L262E;
SEQ ID NO:27+G195E+V199M;
SEQ ID NO:27+S3T+V4I+N261D;
SEQ ID NO:27+A194P+G195E+V199M+V205I;
SEQ ID NO:27+H120D+A228V;
SEQ ID NO:27+S3T+V4I+A228V;
SEQ ID NO:27+H120D+N261D;
SEQ ID NO:27+H120D+S163G+N261D;
SEQ ID NO:27+N76D+A228V+L262E;
SEQ ID NO:27+N76D+Q137H+S141H+R145H+S163G+N238E;
SEQ ID NO:27+Q137H+S141H+R145H+N238E+L262E;
SEQ ID NO:27+S3T+N76D+Q137H+S141H+R145H+S156D+Y209W;
SEQ ID NO:27+H120D+Q137H+S141H+R145H+S163G+N261D;
SEQ ID NO:27+N76D+Q137H+S141H+R145H+A228V+N261D;
SEQ ID NO:27+A194P+G195E+V199M+V205I+A228V+N261D;
SEQ ID NO:27+N62D+H120D;
SEQ ID NO:27+H120D+N261D;
SEQ ID NO:27+N76D+N261D;
SEQ ID NO:27+N76D+A228V+N261D;
SEQ ID NO:27+A194P+G195E+V205I+N261D;
SEQ ID NO:27+N76D+H120D+N261D;
SEQ ID NO:27+H120D+S163G+N261D;
SEQ ID NO:27+S3T+Q59D+N76D;
SEQ ID NO:27+S3T+N76D+H120D;
SEQ ID NO:27+S3T+N76D+A194P+G195E+V199M+V205I;
SEQ ID NO:27+S3T+N76D+S156D;
SEQ ID NO:27+S3T+N76D+Y209W+N261D;
SEQ ID NO:27+S3T+N76D+H120D+Y209W;
SEQ ID NO:27+S3T+N76D+S156D+Y209W;
SEQ ID NO:27+S3T+V4I+N76D+A228V+N261D;
SEQ ID NO:27+S3T+V4I+N76D+H120D;
SEQ ID NO:27+H120D+P131F+A194P+N261D;
SEQ ID NO:27+N76D+E136H+A228V+N261D;
SEQ ID NO:27+N76D+N218S+A228V+N261D;
SEQ ID NO:27+N76D+N218Q+A228V+N261D;
SEQ ID NO:27+N76D+N218A+A228V+N261D;
SEQ ID NO:27+K27Q+R45E;
SEQ ID NO:27+N76D+A228V+L262E;
SEQ ID NO:27+R45E+A88S;
SEQ ID NO:27+S87E+K237E;
SEQ ID NO:27+N261D+L262E;
SEQ ID NO:27+S87E+L262E;
SEQ ID NO:27+S87E+N238E;
SEQ ID NO:27+K27Q+S87E;
SEQ ID NO:27+N76D+N117E;
SEQ ID NO:27+H120D+N238E;
SEQ ID NO:27+Q59D+L262E;
SEQ ID NO:27+K27Q+L262E;
SEQ ID NO:27+H120D+L262E;
SEQ ID NO:27+K27Q+Q59D;
SEQ ID NO:27+K27Q+S156D;
SEQ ID NO:27+K27Q+G61D;
SEQ ID NO:27+Q59D+N261D;
SEQ ID NO:27+Q59D+N117E;
SEQ ID NO:27+K237E+N261D;
SEQ ID NO:27+Q59D+N238E;
SEQ ID NO:27+A15T+H120D+N261D;
SEQ ID NO:27+N76D+S163G+N238E;
SEQ ID NO:27+H120D+S163G+L262E;
SEQ ID NO:27+H120D+S163G+N261D;
SEQ ID NO:27+Q59D+H120D;
SEQ ID NO:27+G61D+N76D;
SEQ ID NO:27+S3T+N76D;
SEQ ID NO:27+S3T+H120D;
SEQ ID NO:27+G61D+H120D;
SEQ ID NO:27+P55S+H120D;
SEQ ID NO:27+S163G+A228V;
SEQ ID NO:27+S163G+N261D;
SEQ ID NO:27+S3T+S163G;
SEQ ID NO:27+G61D+S163G;
SEQ ID NO:27+S156D+S163G;
SEQ ID NO:27+Q59D+S163G;
SEQ ID NO:27+N76D+S163G;
SEQ ID NO:27+P55S+S163G;
SEQ ID NO:27+H120D+S163G;
SEQ ID NO:27+T58L+Q59D;
SEQ ID NO:27+P55S+T58L;
SEQ ID NO:27+T58L+G97D;
SEQ ID NO:27+T58L+S106A;
SEQ ID NO:27+T58L+A228V;
SEQ ID NO:27+S3T+T58L;
SEQ ID NO:27+T58L+S156D;
SEQ ID NO:27+T58L+Y91H;
SEQ ID NO:27+T58L+H120D;
SEQ ID NO:27+T58L+S163G;
SEQ ID NO:27+S163G+N261D;
SEQ ID NO:27+T58L+N261D;
SEQ ID NO:27+T58L+N76D;
SEQ ID NO:27+S3T+N76D+H120D;
SEQ ID NO:27+S3T+N76D+A228V;
SEQ ID NO:27+S3T+N76D+S156D;
SEQ ID NO:27+S3T+N76D+Y209W;
SEQ ID NO:27+S3T+N76D+Y209W+V244T;
SEQ ID NO:27+N76D+H120D;
SEQ ID NO:27+N76D+S156D;
SEQ ID NO:27+H120D S156D;
SEQ ID NO:27+R45E+L262E;
SEQ ID NO:27+Q59D+G61D;
SEQ ID NO:27+S87E+L262E;
SEQ ID NO:27+G61D+L262E;
SEQ ID NO:27+Q59D+L262E;
SEQ ID NO:27+R45E+Q59D;
SEQ ID NO:27+Q59D+S156D;
SEQ ID NO:27+S156D+L262E;
SEQ ID NO:27+S163G+N238E+L262E;
SEQ ID NO:27+S3T+V4I+S163G+N261D;
SEQ ID NO:27+H120D+S163G+N261D;
SEQ ID NO:27+Y91H+N117H+N238H;
SEQ ID NO:27+T58L+S163G+N261D;
SEQ ID NO:27+S3T+V4I+S163G+N261D;
SEQ ID NO:27+S87E+5163G+L262E;
SEQ ID NO:27+S156D+S163G+L262E;
SEQ ID NO:27+T58LS163G+N261D;
SEQ ID NO:27+S156DS163G+L262E;
SEQ ID NO:27+S3T+N76D+Y209W+N261D+L262E;
SEQ ID NO:27+R45E/D/Q;
SEQ ID NO:27+Q58L;
SEQ ID NO:27+Q59D;
SEQ ID NO:27+G61D;
SEQ ID NO:27+S87E;
SEQ ID NO:27+G97S;
SEQ ID NO:27+A98E;
SEQ ID NO:27+N117E;
SEQ ID NO:27+H120D/K/V;
SEQ ID NO:27+P129D;
SEQ ID NO:27+E136Q;
SEQ ID NO:27+Q137H;
SEQ ID NO:27+S156D;
SEQ ID NO:27+S160A;
SEQ ID NO:27+S163A/G;
SEQ ID NO:27+V199M;
SEQ ID NO:27+M222Q;
SEQ ID NO:27+N261 T;
SEQ ID NO:27+L262E/Q/N;
N117E+S3T;
S156D+S3T;
N238E+S3T;
N261D+S3 T;
L262E+S3T;
N117E+N128Q;
S156D+N128Q;
N238E+N128Q;
N261D+N128Q;
L262E+N128Q;
N117E+Q137H;
S156D+Q137H;
N238E+Q137H;
N261D+Q137H;
L262E+Q137H;
N117E+S141H;
S156D+S141H;
N238E+S141H;
N261D+S141H;
L262E+S141H;
N117E+R145H;
S156D+R145H;
N238E+R145H;
N261D+R145H;
L262E+R145H;
N117E+S163G;
S156D+S163G;
N238E+S163G;
N261D+S163G;
L262E+S163G;
N117E+A194P;
S156D+A194P;
N238E+A194P;
N261D+A194P;
L262E+A194P;
N117E+V199M;
S156D+V199M;
N238E+V199M;
N261D+V199M;
L262E+V199M;
N117E+V205I;
S156D+V205I;
N238E+V205I;
N261D+V205I;
L262E+V205I;
N117E+N218Q;
S156D+N218Q;
N238E+N218Q;
N261D+N218Q;
L262E+N218Q;
N117E+A228V;
S156D+A228V;
N238E+A228V;
N261D+A228V;
L262E+A228V;
SEQ ID NO:27+V4D/E/I;
SEQ ID NO:27+R10N/Q/D/E/S;
SEQ ID NO:27+H17D;
SEQ ID NO:27+K27S/N/Q/E/D;
SEQ ID NO:27+R45E/D/Q/N;
SEQ ID NO:27+G53D;
SEQ ID NO:27+Q59D;
SEQ ID NO:27+G61D;
SEQ ID NO:27+L75D;
SEQ ID NO:27+N76D;
SEQ ID NO:27+I79D;
SEQ ID NO:27+S87E;
SEQ ID NO:27+G97D;
SEQ ID NO:27+A98E;
SEQ ID NO:27+*103aE;
SEQ ID NO:27+N117E;
SEQ ID NO:27+H120D;
SEQ ID NO:27+E136K/Q;
SEQ ID NO:27+S156D;
SEQ ID NO:27+R170E/Q/N/D;
SEQ ID NO:27+N185D;
SEQ ID NO:27+G195E;
SEQ ID NO:27+K235L/W/N/Q/E/S;
SEQ ID NO:27+K237N/Q/D/E/S;
SEQ ID NO:27+N238D/E;
SEQ ID NO:27+V244D;
SEQ ID NO: 27+R246Q/E/D;
SEQ ID NO:27+R247S/E;
SEQ ID NO:27+K251S/D/Q/E/N;
SEQ ID NO:27+N261D;
SEQ ID NO:27+L262D/E;
SEQ ID NO:27+S265H;
SEQ ID NO:27+A194P+G195E;
SEQ ID NO:27+G195E+V199M;
SEQ ID NO:27+N76D+A228V+N261D;
SEQ ID NO:27+N76D+S163G+N238E;
SEQ ID NO:27+S156D+L262E;
SEQ ID NO:27+N238E+L262E;
SEQ ID NO:27+S3T+N76D+S156D+Y209W;
SEQ ID NO:27+K27Q+H120D+S163G+N261D;
SEQ ID NO:27+V104T+H120D+S156D+L262E;
SEQ ID NO:27+V104T+S156D+L262E;
SEQ ID NO:27+Q137H+S141H+R145H+N238E+L262E;
SEQ ID NO:27+S3T+V4I+A228V;
SEQ ID NO:27+H120D S163G N261D;
SEQ ID NO:27+N76D+S101E+A228V+L262E;
SEQ ID NO:27+N76D+Q137H+S141H+R145H+S163G+N238E;
SEQ ID NO:27+S3T+N76D+Q137H+S141H+R145H+S156D+Y209W;
SEQ ID NO:27+H120D+Q137H+S141H+R145H+S163G+N261D;
SEQ ID NO:27+A194P+G195E+V199M+V205I;
SEQ ID NO:27+S3T+N76D+A194P+G195E+V199M+V205I;
SEQ ID NO:27+A228V+N261D;
SEQ ID NO:27+N76D+A228V;
SEQ ID NO:27+S3T+V4I+N261D;
SEQ ID NO:27+H120D+A228V;
SEQ ID NO:27+N76D+N261D;
SEQ ID NO:27+A194P+G195E+V199M+V205I+A228V+N261D;
SEQ ID NO:27+A194P+G195E+V205I+A228V; or
SEQ ID NO: 27+H120D+N261D.

3. The cleaning composition of claim 1, wherein the protease is selected from a protease comprising an amino acid sequence having at least 90%, sequence identity to the amino acid sequence set forth in SEQ ID NO:30 over its entire length and comprising an amino acid substitution in at least one position corresponding to positions 12, 43, 122, 127, 154, 156, 160, 211, 212 and 222 of SEQ ID NO:30, wherein the amino acid substitutions are selected from the group consisting of 12L, 43V, 122L, 127P, 154S, 156A, 160S, 211N, 211L, 212D, 212H, and 222S, wherein the numbering is according to SEQ ID NO:30

4. The cleaning composition of claim 1, wherein the protease is selected from a protease comprising an amino acid sequence having at least 90%, sequence identity to the amino acid sequence set forth in SEQ ID NO:39 over its entire length and comprising (i) at least three amino acid substitutions 3T, 4I, 99E and 199I at the positions corresponding to positions 3, 4, 99 and 199 of SEQ ID NO:39 and (ii) in at least one position corresponding to positions 74, 136, 143, 154, 161, 163, 171, 200, 203, 209, 212 or 256 of SEQ ID NO:39.

5. The cleaning composition according to claim 1, wherein the dispersin is microbial.

6. The cleaning composition according to claim 1, wherein the dispersin is obtained from Terribacillus, Curtobacterium, Aggregatibacter, Haemophilus, Actinobacillus, Lactobacillus, Staphylococcus, Neisseria, Otariodibacter, Lactococcus, Frigoribacterium, Basfia, Weissella, Macrococcus, or Leuconostoc.

7. The cleaning composition according to claim 1, wherein the dispersin catalyzes the hydrolysis of β-1,6-glycosidic linkages of N-acetyl-glucosamine polymers.

8. The cleaning composition according to claim 5, wherein the dispersin comprises a polypeptide selected from the group consisting of;
i) a polypeptide having at least 90%, sequence identity to the amino acid sequence shown in SEQ ID NO: 1;
ii) a polypeptide having at least 90% sequence identity to the amino acid sequence shown in SEQ ID NO: 2;
iii) a polypeptide having at least 90% sequence identity to the amino acid sequence shown in SEQ ID NO: 3;
iv) a polypeptide having at least 90% sequence identity to the amino acid sequence shown in SEQ ID NO: 4;
v) a polypeptide having at least 90% sequence identity to the amino acid sequence shown in SEQ ID NO: 5;
vi) a polypeptide having at least 90% sequence identity to the amino acid sequence shown in SEQ ID NO: 6;
vii) a polypeptide having at least 90% sequence identity to the amino acid sequence shown in SEQ ID NO: 7;
viii) a polypeptide having at least 90% sequence identity to the amino acid sequence shown in SEQ ID NO: 8;
ix) a polypeptide having at least 90% sequence identity to the amino acid sequence shown in SEQ ID NO: 9;
x) a polypeptide having at least 90% sequence identity to the amino acid sequence shown in SEQ ID NO: 10;
xi) a polypeptide having at least 90% sequence identity to the amino acid sequence shown in SEQ ID NO: 11;
xii) a polypeptide having at least 90% sequence identity to the amino acid sequence shown in SEQ ID NO: 12;
xiii) a polypeptide having at least 90% sequence identity to the amino acid sequence shown in SEQ ID NO: 13;
xiv) a polypeptide having at least 90% sequence identity to the amino acid sequence shown in SEQ ID NO: 14;
xv) a polypeptide having at least 90% sequence identity to the amino acid sequence shown in SEQ ID NO: 15;
xvi) a polypeptide having at least 90% sequence identity to the amino acid sequence shown in SEQ ID NO: 16;
xvii) a polypeptide having at least 90% sequence identity to the amino acid sequence shown in SEQ ID NO: 17;
xviii) a polypeptide having at least 90% sequence identity to the amino acid sequence shown in SEQ ID NO: 18;
xix) a polypeptide having at least 90% sequence identity to the amino acid sequence shown in SEQ ID NO: 19;
xx) a polypeptide having at least 90% sequence identity to the amino acid sequence shown in SEQ ID NO: 20;
xxi) a polypeptide having at least 90% sequence identity to the amino acid sequence shown in SEQ ID NO: 21;
xxii) a polypeptide having at least 90% sequence identity to the amino acid sequence shown in SEQ ID NO: 22; and
xxiii) a polypeptide having at least 90% sequence identity to the amino acid sequence shown in SEQ ID NO: 23.

9. The cleaning composition according to claim 1, wherein the dispersin comprises a polypeptide having at least 95% sequence identity to the amino acid sequence shown in SEQ ID NO: 17.

10. The cleaning composition according to claim 1, wherein an amount of dispersin in the composition ranges from 0.01 to 1000 ppm and the amount of protease ranges from 0.01 to 1000 ppm.

11. The cleaning composition according to claim 1, wherein the cleaning component is selected from surfactants, builders, and bleach components.

12. The cleaning composition of claim 1, wherein the composition
(a) is a solid, and further comprises
(a1) at least one zeolite builder;
(a2) at least one phosphonate builder;
(a3) at least one further enzyme; and
(a4) at least one polymer; or
(b) is a solid laundry detergent composition and further comprises
(b1) at least one silicate builder;
(b2) optionally carboxymethylcellulose;
(b3) at least one further enzyme;
(b4) optionally at least one soil release polymer; and
(b5) at least one bleaching system, comprising a bleaching agent, a bleach activator, and a bleach catalyst; or
(c) is liquid laundry detergent composition and further comprises
(c1) at least one surfactant;
(c2) optionally at least one phosphonate builder;
(c3) optionally at least one further enzyme; and
(c4) optionally at least one organic solvent; or
(d) is a liquid laundry detergent in unit dose form, and further comprises
(d1) water in an amount of up to 20 wt.-%;
(d2) optionally at least one bittering agent;
(d3) optionally at least one optical brightener; and
(d4) optionally at least one polymer; or
(e) is a fabric finisher and further comprises
(e1) at least one softening silicone;
(e2) at least one perfume;
(e3) optionally polyquaternium 10 in an amount ranging from 0.1 to 20 wt.-%;
(e4) optionally polyquaternium 37 in an amount ranging from 0.1 to 20 wt.-%;
(e5) optionally a plant-based esterquat; and
(e6) optionally adipic acid, in an amount ranging from 0.1 to 20 wt.-%; or
(f) is an acidic cleaning agent, and further comprises
(f1) plant-based or bio-based surfactants;
(f2) at least one acidic biocide; and
(f3) at least one soil release, water repellant, or water spreading polymer; or
(g) is a neutral cleaning agent, and further comprises
(g1) plant-based or bio-based surfactants;
(g2) at least one biocide; and
(g3) at least one soil release, water repellant or water spreading polymer; or
(h) is an alkaline cleaning agent, and further comprises
(h1) plant-based or bio-based surfactants; or
(i) is a hand dishwashing agent, and further comprises
(i1) at least one anionic surfactant;
(i2) at least one amphoteric surfactant;
(i3) at least one nonionic surfactant;
(i4) at least one further enzyme; or
(j) is an automatic dishwashing composition and further comprises
(j1) at least one builder selected from citrate, aminocarboxylates, and combinations thereof;
(j2) at least one phosphonate builder;
(j3) at least one nonionic surfactant;
(j4) at least one bleaching system comprising a bleaching agent, a bleach activator, and a bleach catalyst; and
(j5) at least one polymer selected from sulfopolymers, cationic polymers, and polyacrylates; or
(k) further comprises
(k1) at least one sulfopolymer; or
(l) further comprises probiotics; or
(m) is in unit dose form and comprises at least 2 separate compartments; or
(n) is a phosphate-free composition.

13. (canceled)

14. A method of deep cleaning an item, wherein the method comprise:
a) contacting the item with a cleaning composition according to claim 1; and
b) rinsing the item, wherein the item is a textile.

15. The cleaning composition according to claim 5, wherein the dispersion is obtained from bacteria or fungi.
